(19) United States
(12) Patent Application Publication
(10) Pub. No.: US 2008/0058673 A1
(43) Pub. Date: Mar. 6, 2008

(54) Tissue extraction device and method of using the same

(76) Inventors: Lex Jansen, Pleasanton, CA (US); Art Ferdinand, San Jose, CA (US); Hugues F. Malandain, Mountain View, CA (US); Claudia Orellana, San Jose, CA (US); Chris Phan, San Leandro, CA (US); Derek Rothwell, Los Altos, CA (US); Karen D. Talmadge, Los Altos Hills, CA (US); Jenifer Wong, Sunnyvale, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 11/511,529

(22) Filed: Aug. 29, 2006

(51) Int. Cl. A61B 10/02

(52) U.S. Cl. 600/567

(57) ABSTRACT

Apparatuses for performing minimally invasive medical procedures are disclosed herein. In one example, an apparatus includes an outer body that defines an opening at a distal end, and a cutting portion disposed at an edge of the opening. An inner body is movably disposed within the outer body and defines an opening at a distal end. The outer body and the inner body collectively have a first configuration in which the opening on the outer body is substantially aligned with the opening on the inner body and the opening on the outer body is configured to receive a portion of a tissue, and a second configuration in which the opening on the outer body and the opening on the inner body are substantially misaligned and the cutting portion on the outer body is configured to sever the portion of the tissue from a remaining portion of the tissue.

### BACKGROUND

The invention relates generally to medical devices and procedures, and more particularly to a medical device for accessing percutaneously a tissue and taking a biopsy of a tissue, such as a tumor, within the tissue.

Known medical devices are configured to access percutaneously a tissue, such as a vertebra or other area of a spine, to perform a variety of different medical procedures. Some known medical devices are configured to remove tissue from within the interior of a vertebra or intervertebral disc. Other known medical devices are configured to provide some type of cutting means to tear or loosen tissue within a vertebra or intervertebral disc.

There are also a variety of medical devices configured to remove a portion of bone or tissue, such as a portion of a tumor, from within a bone structure or soft tissue area of a patient's body. An example of such a device is a biopsy needle. A problem with many biopsy-type medical devices is that the device can cut or sever the biopsy sample, but may have difficulty retaining the biopsy sample within the device during removal from the patient. In addition, some biopsy devices cannot penetrate hard or solid tumors, such as those sometimes found in bone structures, such as a vertebra.

Thus, a need exists for an apparatus and method for removing a portion of a hard or soft tissue from within a bone structure, such as a vertebra, or soft tissue area, with minimal damage to the surrounding bone or soft tissue.

### SUMMARY OF THE INVENTION

Apparatuses for performing minimally invasive medical procedures are disclosed herein. In one example, an apparatus includes an outer body that defines an opening at a distal end and a cutting portion disposed at an edge of the opening. An inner body is movably disposed within the outer body. The inner body defines an opening at a distal end of the inner body. The outer body and the inner body collectively have a first configuration in which the opening on the outer body is substantially aligned with the opening on the inner body and the opening on the outer body is configured to receive a portion of a tissue. The outer body and the inner body collectively have a second configuration in which the opening on the outer body and the opening on the inner body are substantially misaligned and the cutting portion on the outer body is configured to sever the portion of the tissue from a remaining portion of the tissue.

### BRIEF DESCRIPTION OF THE DRAWINGS

The present invention is described with reference to the accompanying drawings.

FIG. 1 is a schematic illustration of a medical device according to an embodiment of the invention.

FIG. 2 is a side perspective view of a portion of a medical device in an open configuration according to an embodiment of the invention.

FIG. 3 is a top view of the medical device of FIG. 2 shown in a substantially closed configuration.

FIG. 4 is a side view of the medical device of FIG. 2 shown inserted at least partially into a tumor within a vertebra in the open configuration.

FIG. 5 is a cross-sectional view taken along line 5-5 in FIG. 3.

FIG. 6 is a cross-sectional view taken along line 6-6 in FIG. 2

FIG. 7 through FIG. 12 are each a side perspective view of a portion of a medical device according various embodiments of the invention.

FIG. 13 is a side perspective view of a portion of a medical device according to an embodiment of the invention shown partially in cross-section.

FIG. 14 is a side perspective view of a portion of a medical device according to an embodiment of the invention.

FIG. 15 is a side perspective view of a portion of the medical device of FIG. 14 shown in an expanded configuration.

FIG. 16 is a side perspective view of a portion of the medical device of FIG. 15 shown in a collapsed configuration.

FIG. 17 is a cross-sectional view taken along the line 17-17 in FIG. 14.

FIG. 18 is a cross-sectional view taken along the line 18-18 in FIG. 14.

FIG. 19 is a cross-sectional view illustrating an alternative embodiment of an elongate body.

FIG. 20 is a side view of a portion of the medical device of FIG. 14 in an expanded configuration.

FIG. 21 is a distal end view of the portion of a medical device of FIG. 20 shown in an expanded configuration.

FIG. 22 is a distal end view of the portion of a medical device of FIG. 20 shown in a collapsed configuration.

FIG. 23A is a perspective view of a portion of a medical device according to another embodiment of the invention in an expanded configuration.

FIG. 23B is a side view of the portion of the medical device of FIG. 23A in an expanded configuration.

FIG. 24 is a distal end view of the portion of the medical device of FIG. 23A shown in an expanded configuration.

FIG. 25 is a distal end view of the portion of the medical device of FIG. 23A shown in a collapsed configuration.

FIG. 26 is a side perspective view of a portion of a medical device according to an embodiment of the invention.

FIG. 27 is a side perspective view of a portion of a medical device according to an embodiment of the invention shown in an open configuration.

FIG. 28 is a side view of the portion of the medical device of FIG. 27 shown in a substantially closed configuration.

FIG. 29 is a side perspective view of a portion of a medical device according to an embodiment of the invention shown in an open configuration.

FIG. 30 is a side perspective view of the portion of a medical device shown in FIG. 29 shown in a substantially closed configuration.

FIG. 31 is an end view taken along line 31-31 in FIG. 29.

FIG. 32 is an end view taken along line 32-32 in FIG. 30.

FIG. 33 is a side view of a portion of a medical device according to an embodiment of the invention shown in an extended configuration.

FIG. 34 is a side view of the portion of the medical device of FIG. 33 shown in a collapsed configuration.

FIG. 35 is a side view of a portion of a medical device according to an embodiment of the invention.

FIG. 36 is a side view shown partially in cross-section of a portion of the medical device of FIG. 35 shown in a collapsed configuration.

FIG. 37 is a side view of the medical device of FIG. 35 shown in an expanded configuration in various positions within a vertebra.

FIG. 38 is a side view in partial cross-section of a portion of a medical device according to an embodiment of the invention.

FIG. 39 is a side view in partial cross-section of a portion of a medical device according to an embodiment of the invention.

FIG. 40 is a side view in partial cross-section of a portion of a medical device according to an embodiment of the invention.

FIG. 41 is a side view of a portion of a medical device according to an embodiment of the invention.

FIG. 42 is an end view taken along line 42-42 in FIG. 41

FIG. 43 is a side view of a portion of a medical device according to an embodiment of the invention.

FIG. 44 is a side view of a portion of a medical device according to an embodiment of the invention.

FIG. 45 is a side cross-sectional view of the portion of a medical device shown in FIG. 44.

FIG. 46 is a side cross-sectional view of a portion of a medical device according to an embodiment of the invention.

FIG. 47 is a side view of a portion of a medical device according to an embodiment of the invention shown in a substantially open configuration.

FIG. 48 is a side view of the portion of a medical device of FIG. 47 shown in a substantially closed configuration.

FIG. 49 is a cross-sectional view taken along line 49-49 in FIG. 47.

FIG. 50 is a side view of a portion of a medical device according to an embodiment of the invention.

FIG. 51 is a distal end view of the medical device of FIG. 50 in a collapsed or substantially closed configuration.

FIG. 52 is a distal end view of the medical device of FIG. 50 in an expanded or open configuration.

FIG. 53 is a side view partially in cross-section of a portion of the medical device of FIG. 50 shown in an expanded or open configuration and partially inserted into a tumor.

FIG. 54 is a side view partially in cross-section of a portion of the medical device of FIG. 50 shown in a collapsed or substantially closed configuration and removed from the tumor.

FIG. 55 is a side cross-sectional view of a portion of a medical device according to an embodiment of the invention shown in an expanded configuration in a portion of a tumor.

FIG. 56 is a side cross-sectional view of a portion of the medical device of FIG. 55 shown in a collapsed configuration.

FIG. 57 is a side cross-sectional view of a portion of a medical device according to an embodiment of the invention shown in an expanded configuration in a portion of a tumor.

FIG. 58 is a side cross-sectional view of a portion of the medical device of FIG. 57 shown in a collapsed configuration in a portion of a tumor.

FIG. 59 is a side view shown partially in cross-section of a medical device according to an embodiment of the invention shown inserted partially within a vertebra.

FIGS. 60-66 are each flowcharts illustrating methods according to various embodiments of the invention.

### DETAILED DESCRIPTION

The medical devices described herein are configured for percutaneous deployment within an interior area of a patient's body, such as within a bone structure or soft tissue area of a patient to remove or biopsy a portion of a tissue, such as a tumor, within the bone structure or soft tissue area. For example, a medical device according to an embodiment of the invention includes a cutting portion that can sever a portion of a tumor within a bone structure or soft tissue area of the patient. In some embodiments, the medical device includes an apparatus having an outer body and an inner body movably disposed within the outer body. In other embodiments, a medical device includes only a single elongate body. In some embodiments, an outer body and/or an inner body can have an expanded configuration and a collapsed configuration, an open and a closed configuration, or an extended and a collapsed configuration.

In one embodiment, an apparatus includes an outer body that defines an opening at a distal end and a cutting portion disposed at an edge of the opening. An inner body is movably disposed within the outer body. The inner body defines an opening at a distal end of the inner body. The outer body and the inner body collectively have a first configuration in which the opening on the outer body is substantially aligned with the opening on the inner body and the opening on the outer body is configured to receive a portion of a tissue. The outer body and the inner body collectively have a second configuration in which the opening on the outer body and the opening on the inner body are substantially misaligned and the cutting portion on the outer body is configured to sever the portion of the tissue from a remaining portion of the tissue.

In another embodiment, an apparatus includes an elongate body that defines a lumen and a cutting portion extending across at least a portion of the lumen of the elongate body to define a first opening and a second opening each in communication with the lumen of the elongate body. The elongate body is configured to be inserted at least partially into a tissue such that a portion of the tissue is cut by the cutting portion and disposed within the lumen of the elongate body. The elongate body is configured to be rotated and moved proximally such that the portion of the tissue is severed from the remaining portion of the tissue.

The term “cutting portion” is used here to mean a component of the apparatus having at least one cutting surface and being configured to disrupt tissue (e.g., a tumor, aggregate of cells, etc.). The cutting portion can be, for example, a cutting surface disposed on an elongate body, such as a cutting surface disposed on an edge of an opening on the elongate body. The cutting portion can also be disposed on, for example, a portion of an elongate body having a collapsed configuration and an expanded configuration.

The term “cannula” is used here to mean a component of the apparatus having one or more passageways configured to receive a device therethrough. For example, a cannula can provide access through a passageway to an interior portion of a tissue or organ. A cannula can be substantially hollow and/or tubular. A cannula can be a variety of different shapes and size. For example, a cannula can have an outer perimeter and/or inner diameter that is, for example, round, square, rectangular, triangular, oval, octagonal, etc.

The term “tissue” is used here to mean any aggregation of cells. For example, a tissue can be a soft tissue area (e.g., a muscle), a hard tissue area (e.g., a bone structure), a vertebral body, an intervertebral disc, a tumor, etc.

As used in this specification and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, an end of a medical device inserted inside the patient's body would be the distal end of the medical device, while the end of the medical device outside the patient's body would be the proximal end of the medical device.

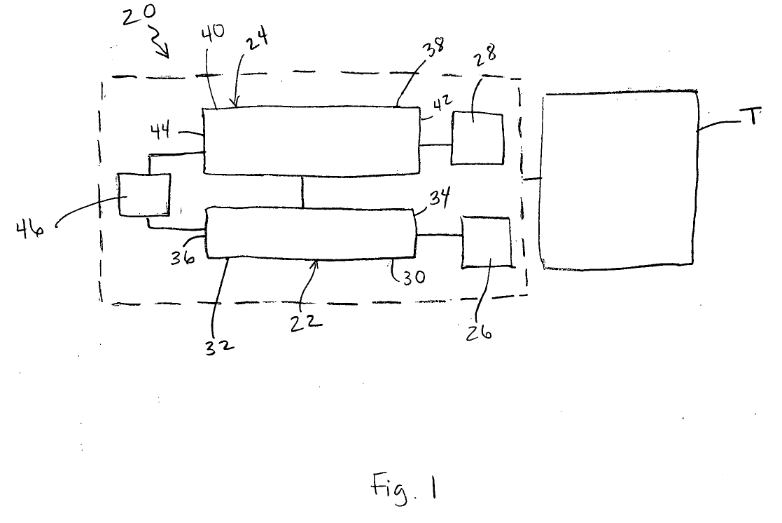

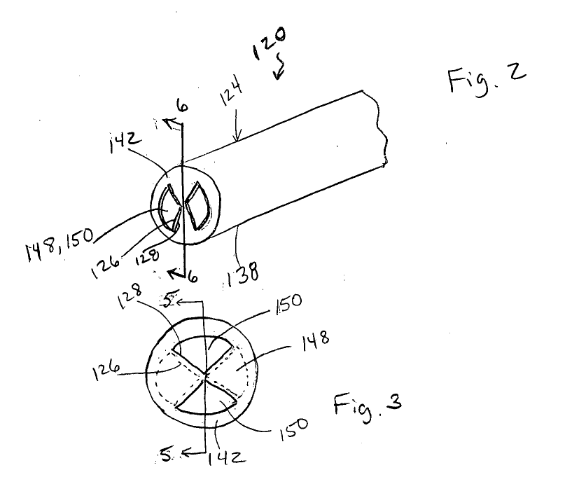

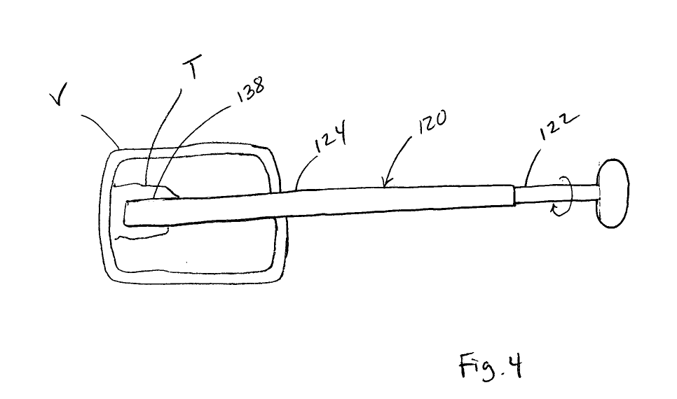

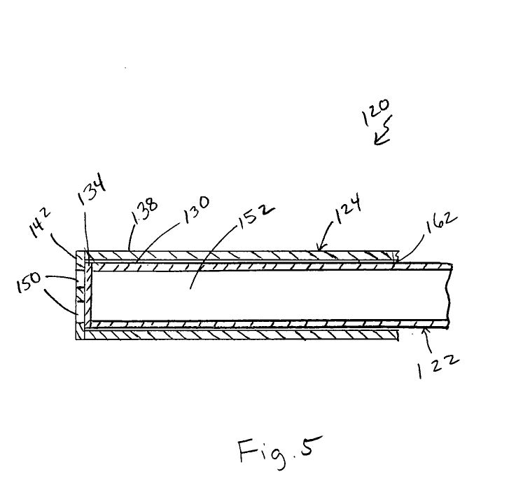

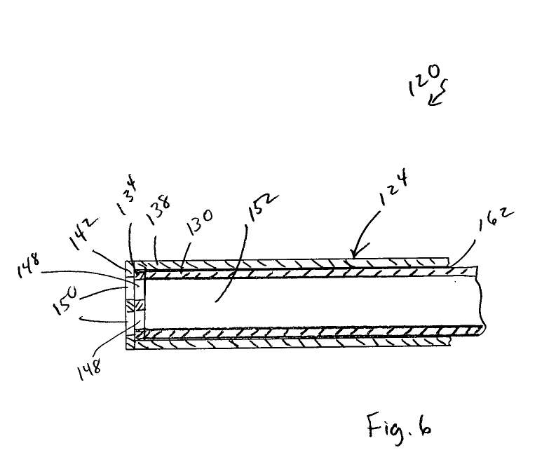

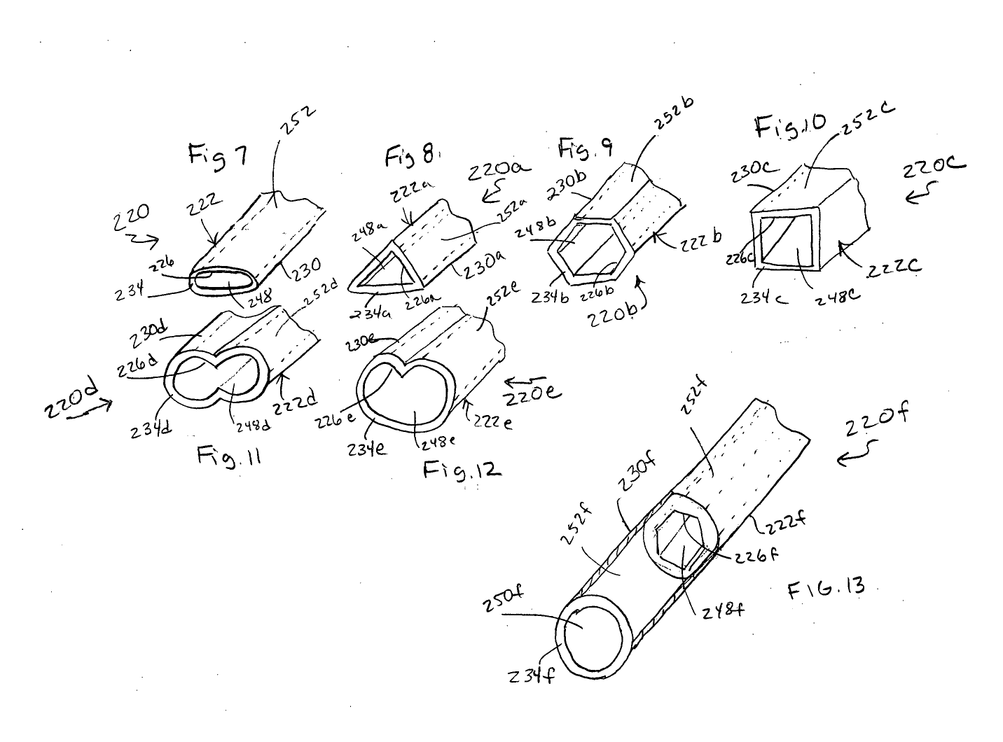

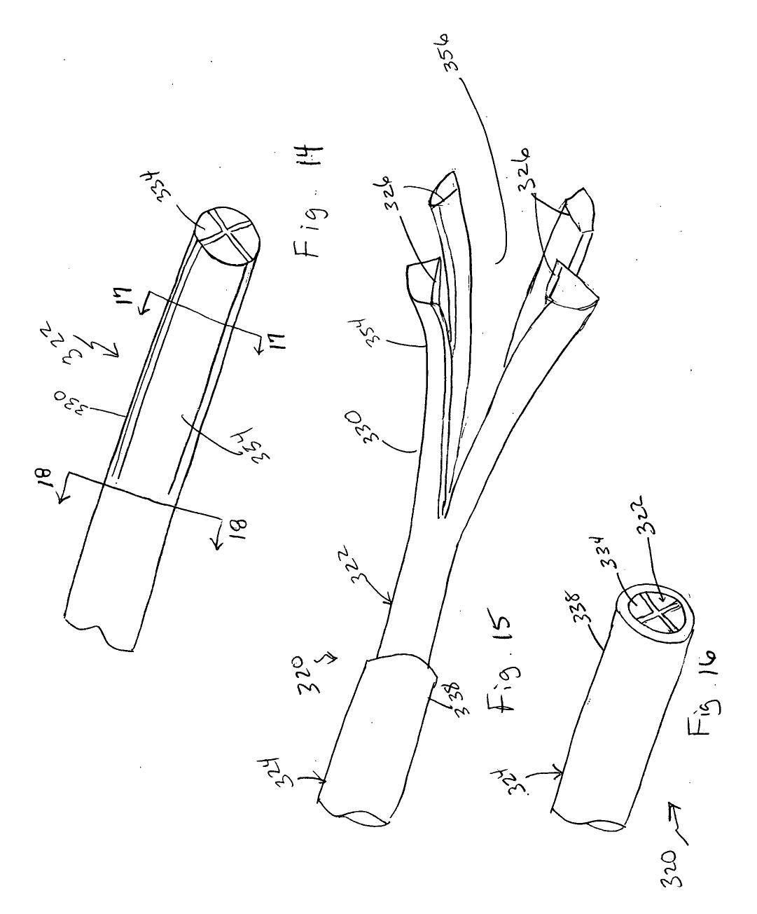

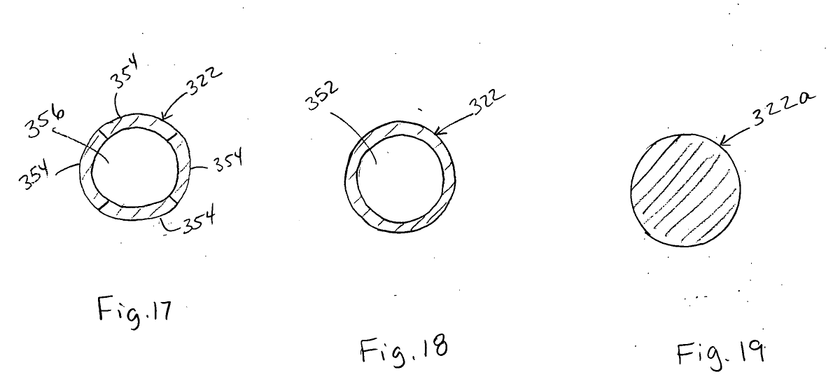

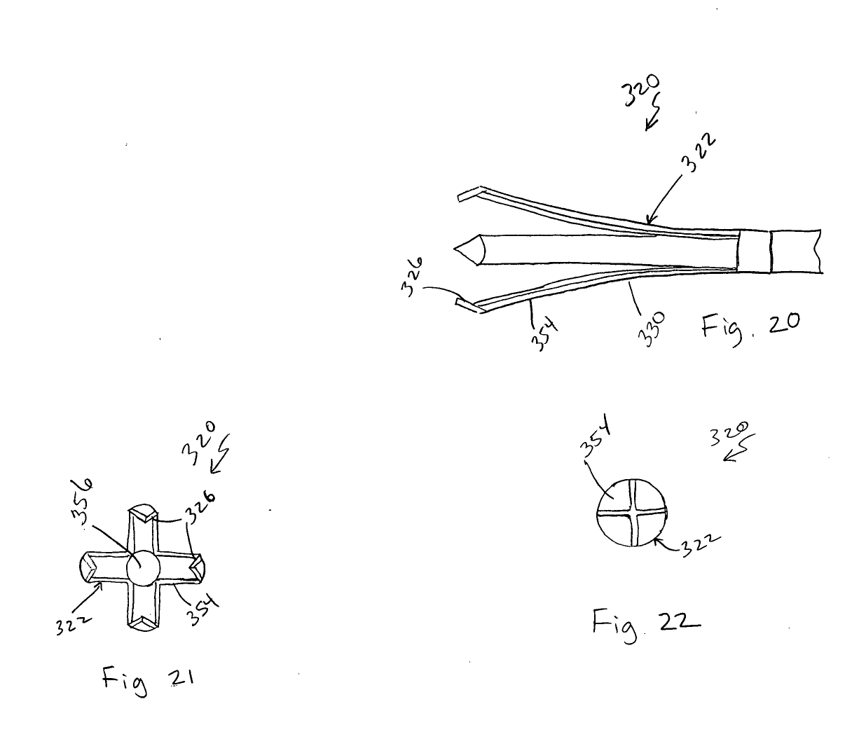

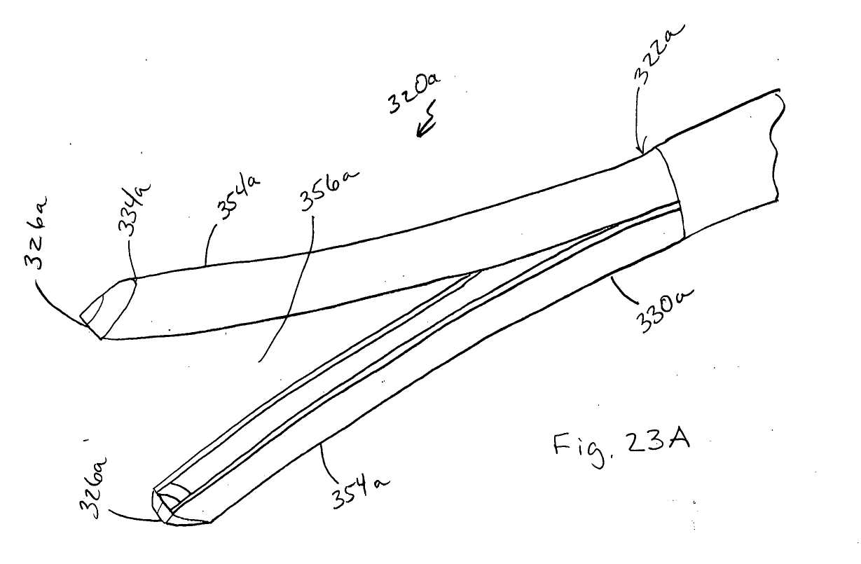

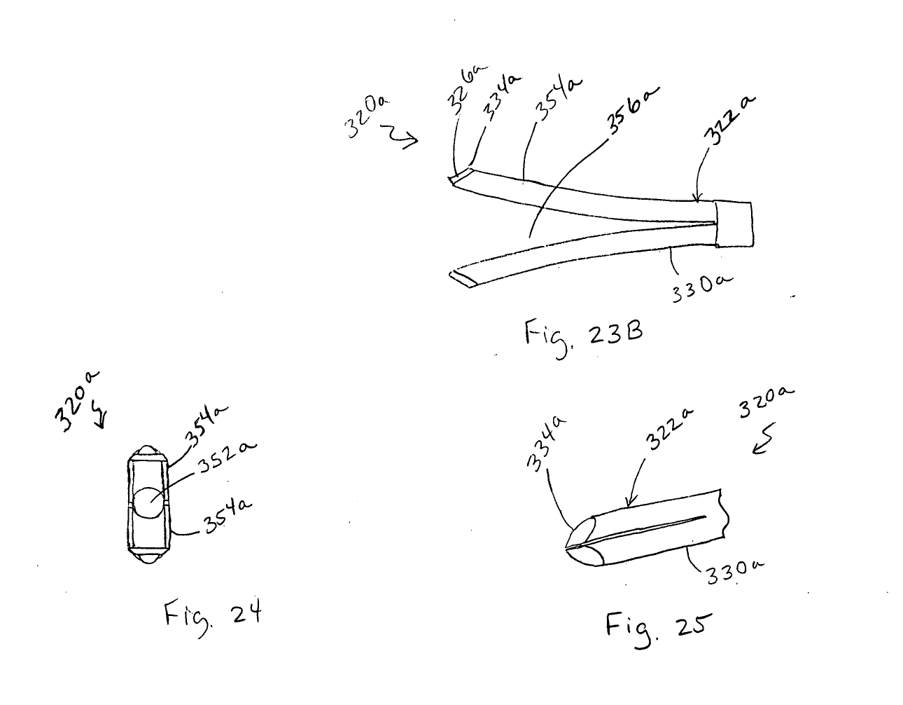

FIG. 1 is a schematic illustration of a medical device 20 and a vertebra V. A medical device 20 according to an embodiment of the invention can include a cannula or outer body 24 and an elongate body or inner body 22 having a cutting portion 26. In some embodiments, a medical device is configured with only an elongate body. The cannula or outer body 24 can also include a cutting portion 28. The cutting portion 26 and/or the cutting portion 28 can be configured to cut or sever a portion of a tissue within a bone structure or soft tissue area of a patient, such as a tumor within a vertebra. The cutting portion 26 and the cutting portion 28 can each include a variety of different configurations, shapes and sizes depending on the particular configuration of the medical device 20. For example, in one embodiment, the cutting portion 26 and/or the cutting portion 28 can each be disposed at an edge of an opening. In other embodiments, the cutting portion 26 can be disposed at an inner surface of a lumen (not shown in FIG. 1) defined by the elongate body 22 or on an outer surface of the elongate body 22. Likewise, the cutting portion 28 can be disposed at an inner surface and within a lumen of the cannula 24 or on an outer surface of the cannula 24.

The elongate body or inner body 22 (sometimes referred to only as elongate body 22 or only as inner body 22 for simplicity) can include a distal portion 30 having a distal end 34, and a proximal portion 32 having a proximal end 36. The elongate body 22 can also include a lumen (not shown in FIG. 1) between the proximal portion 32 and the distal portion 30. In some embodiments, a lumen extends from the distal end 34 to the proximal end 36 of the elongate body 22. In other embodiments, the lumen is disposed within only a portion of the elongate body 22.

The cannula or outer body 24 (sometimes referred to only as cannula 24 or only as outer body 24 for simplicity) includes a distal portion 38 having a distal end 42, a proximal portion 40 having a proximal end 44, and defines a lumen (not shown in FIG. 1) between the proximal portion 40 and the distal portion 38. In some embodiments, the elongate body 22 can be configured to be movably disposed within the lumen of the cannula 24. The medical device 20 can also include an optional seal member 46 coupled to the elongate body 22 and/or the cannula 24. The seal member 46 is configured to seal a space between an outer surface of the elongate body 22 and an inner surface of the cannula 24.

The medical device 20 is configured to be percutaneously inserted into a bone structure, such as a vertebra, or soft tissue area of a patient, such that the cutting portion 26 and/or cutting portion 28 is positioned adjacent to, in contact with, or inserted partially within a tumor within the bone structure or soft tissue area. The cutting portion 26 and/or the cutting portion 28 is configured to sever a portion of the tissue, such as a portion of a tumor, and the medical device 20 is configured to remove the severed portion of the tissue from the remaining portion of the tissue within the bone structure or soft tissue area.

The following embodiments are examples of various embodiments of the medical device 20. The embodiments described below are described with reference to use within a vertebra for simplicity, but it is to be understood that the medical devices described herein can be used on other areas within a patient's body. The embodiments are also described with reference to removing a portion of a tumor, but it is to be understood that the methods and apparatuses described herein can be used to remove a portion of other types of tissue.

FIGS. 2-6 illustrate a medical device 120 according to an embodiment of the invention. The medical device 120 includes an outer body 124 and an inner body 122 movably disposable within the outer body 124. The outer body 124 has a proximal portion (not shown) and a distal portion 138 having a distal end 142. The outer body 124 also defines a lumen 162 (FIGS. 5 and 6) and defines one or more openings 150 (e.g., two openings 150 are shown in FIGS. 2 and 3) on the distal end 142 that are in communication with the lumen 162 of the outer body 124. A cutting portion 128 can be disposed on the outer body 124 at one or more edges of one or more of the openings 150.

The inner body 122 includes a proximal portion (not shown) and a distal portion 130 having a distal end 134. The inner body 122 also defines a lumen 152 and defines one or more openings 148 on the distal end 134 of the inner body 122 that are in communication with the lumen 152 of the inner body 122.

The medical device 120 has an open configuration and a closed configuration. In the open configuration, as shown in FIGS. 2 and 6, the openings 150 on the distal end 142 of the outer body 124 are substantially aligned with the openings 148 on the distal end 134 of the inner body 122. In the closed configuration, the openings 150 on the distal end 142 of the outer body 124 are substantially misaligned with the openings 148 on the distal end 134 of the inner body 122. FIGS. 3 and 5 show an example of the closed configuration where the openings 150 on the distal end 142 of the outer elongate body 124 are entirely misaligned with the openings 148 on the distal end 134 of the inner elongate body 122.

In one use, the medical device 120 is configured to be percutaneously inserted into a vertebra while the medical device is in the open configuration such that the distal end 142 of the outer body 124 is inserted into a tumor T within the vertebra V as shown in FIG. 4. In some embodiments, the medical device 120 is initially inserted into the cortical bone of a vertebra in the closed configuration, and then moved to the open configuration as the medical device 120 is moved into the cancellous bone of the vertebra. In the open configuration and while inserted in the tumor T, a portion of the tumor T will be disposed within the lumen 152 of the inner body 122. The inner body 122 can then be rotated within the outer body 124 such that the openings 148 and the openings 150 become substantially misaligned and the medical device 120 is moved to the closed configuration. When the inner body 122 is rotated within the outer body 124, the cutting portion 128 and the cutting portion 126 can sever the portion of the tumor T that is disposed within the lumen 152 of the inner body 122 from the remaining portion of the tumor T. Although this embodiment illustrates a cutting portion on both the inner body 122 and the outer body 124, other embodiments may only have a cutting portion 126 or a cutting portion 128. In other embodiments, more than one cutting portion 126 can be disposed on the inner body 122 and/or more than one cutting portion 128 can be disposed on the outer body 124.

FIGS. 7 through 13 illustrate various alternative embodiments of a portion of a medical device according to the invention. Such embodiments include an opening for receiving a portion of a tissue that is non-circular. As will be described below, the non-circular opening enables the medical device to cut or sever a portion of a tissue by rotating the medical device. A medical device 220 (220a, 220b, 220c, 220d, 220e, 220f) includes an elongate body 222 (222a, 222b, 222c, 222d, 222e, 222f) having a distal portion 230 (230a, 230b, 230c, 230d, 230e, 230f) and a distal end 234 (234a, 234b, 234c, 234d, 234e, 234f), and defines a lumen 252 (252a, 252b, 252c, 252d, 252e, 252f). Each of the elongate bodies 222 (222a, 222b, 222c, 222d, 222e, 222f) define an opening 248 (248a, 248b, 248c, 248d, 248e, 248f) on the distal portion 230 (230a, 230b, 230c, 230d, 230e, 230f) where the opening has a non-circular cross-section and is in communication with the lumen 252 (252a, 252b, 252c, 252d, 252e, 252f).

In the embodiments shown in FIGS. 7-12, the opening 248 (248a, 248b, 248c, 248d, 248e) is disposed at the distal end 234 (234a, 234b, 234c, 234d, 234e). In the embodiment shown in FIG. 13, the opening 248f is disposed at a distance from the distal end 238f. Thus, the opening 248 for any of the embodiments can be disposed at a distal end 234 of the elongate body 222 or at a distance from the distal end 234 of the elongate body 222 within the lumen 152 of the elongate body. In an embodiment such as medical device 220f, the elongate body 222f defines an opening 250f disposed at the distal end 234F that is also in communication with the lumen 252f.

The medical device 220 (220a, 220b, 220c, 220d, 220e, 220f) also includes a cutting portion 226 (226a, 226b, 226c, 226d, 226e, 226f) disposed at at least a portion of at least one edge of the opening 248 (248a, 248b, 248c, 248d, 248e, 248f). In some embodiments, a cutting portion (not shown) can also be disposed at at least a portion of an edge of the opening 250f.

In use, the medical device 220 (220a, 220b, 220c, 220d, 220e, 220f) is inserted into a vertebra (e.g., through the annulus of the vertebra) and at least partially into a tumor within the vertebra. When the medical device 220 (220a, 220b, 220c, 220d, 220e, 220f) is inserted into the tumor, a portion of the tumor will be disposed within the lumen 252 (252a, 252b, 252c, 252d, 252e, 252f) of the elongate body 222 (222a, 222b, 222c, 222d, 222e, 222f). The elongate body 222 (222a, 222b, 222c, 222d, 222e, 222f) can then be rotated and the cutting portion 226 (226a, 226b, 226c, 226d, 226e, 226f) combined with the non-circular cross-section of the opening 248 (248a, 248b, 248c, 248d, 248e, 248f) will sever the portion of the tumor disposed within the lumen 252 (252a, 252b, 252c, 252d, 252e, 252f) from the remaining portion of the tumor within the vertebra.

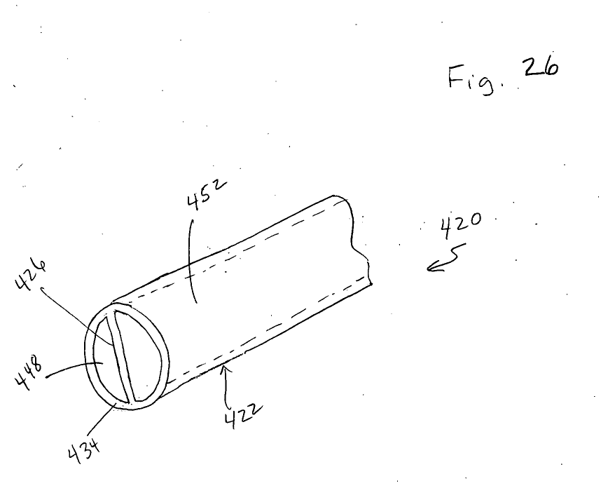

FIGS. 14-22 illustrate another medical device according to an embodiment of the invention having an expanded and a collapsed configuration, as will be described below. A medical device 320 includes a cannula 324 and an elongate body 322 movably disposed within a lumen (not shown in FIGS. 14-22) of the cannula 324 between a distal portion 338 and a proximal portion (not shown in FIGS. 14-22).

The elongate body 322 includes a proximal portion (not shown) and a distal portion 330 having a distal end 334. The distal portion 330 of the elongate body 322 defines a set of four arms 354 that can, in some embodiments, include a cutting portion 326 on the distal end 334 of the elongate body 322. The set of arms 354 can be formed, for example, by cutting slits along a wall of the elongate body 322. The number of arms can vary depending on the particular embodiment. The set of arms 354 define an interior volume 356; the elongate body 322 can, in some embodiments define a lumen 352 as shown in FIG. 17. In some embodiments, the elongate body does not define a lumen as shown in the alternative embodiment of an elongate body 322a illustrated in FIG. 19.

The elongate body 322 has an open or expanded configuration, as shown in FIGS. 15, 20 and 21 and a collapsed or closed configuration, as shown in FIGS. 14, 16 and 22. For example, the set of arms 354 can be constructed with an elastic or super-elastic material such that the set of arms 354 are biased into an open or expanded configuration when not constrained, as shown in FIG. 15. An example of a super-elastic material is Nitinol, and examples of elastic material include various cobalt alloys. The cannula 324 can be moved over the distal portion 330 of the elongate body 322 to move the elongate body 322 to the collapsed or closed configuration, as shown in FIG. 16. When the elongate body 322 is in the closed or collapsed configuration, the interior volume 356 defined by the set of arms 354 is smaller (see FIG. 17) than the interior volume 356 defined by the set of arms 354 when the elongate body 322 is in the open or expanded configuration (see FIG. 15).

In use, the medical device 320 is percutaneously inserted into a vertebra with the elongate body 322 in the collapsed configuration. The distal portion 330 of the elongate body 322 is then moved distally to a location outside of the cannula 324 and within the interior of the vertebra. This moves the elongate body 322 to the expanded configuration with the set of arms 354 biased to an open configuration and positioned adjacent to, or in contact with, a tumor within the vertebra. For example, the set of arms 354 can be positioned such that at least a portion of the tumor is positioned at least partially within the interior volume 356 defined by the set of arms 354. To sever the portion of the tumor from the remaining portion of the tumor, the cannula 324 can be moved distally over the distal portion 330 of the elongate body 322, or alternatively, the distal portion 330 of the elongate body 322 can be moved proximally such that it is moved to a position within the lumen of the cannula 324. In either case, the elongate body 322 is moved to the collapsed configuration and the portion of the tumor is severed from the remaining portion of the tumor with the cutting portions 326. The severed portion of the tumor will then be within the interior volume 326 of the set of arms 354, which is collapsed within the lumen of the cannula 324.

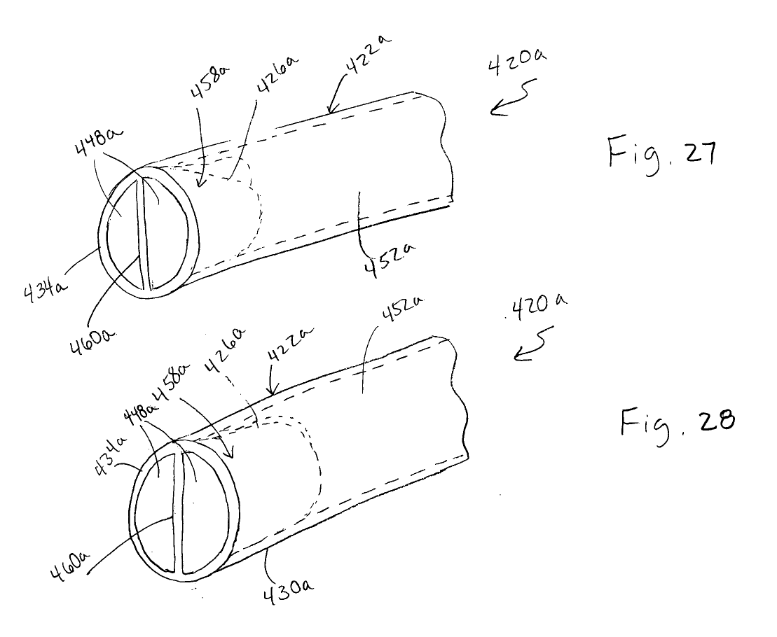

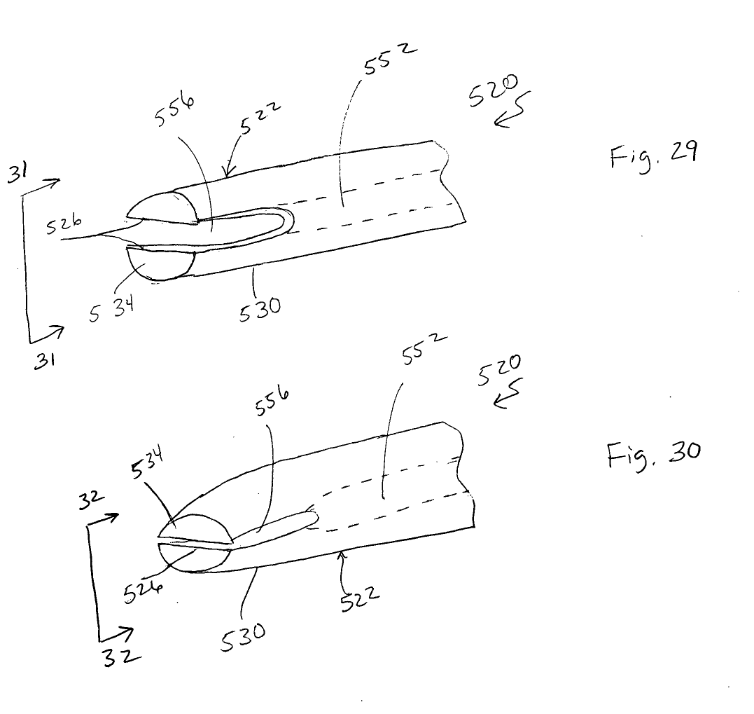

FIGS. 23A-25 illustrate a portion of another embodiment of a medical device that is similar to the medical device 320. A medical device 320a includes a cannula (not shown) and an elongate body 322a movably disposed within a lumen (not shown) of the cannula. In this embodiment, the elongate body 322a includes a proximal portion (not shown) and a distal portion 330a having a distal end 334a. The distal portion 330a of the elongate body 322a defines a set of two arms 354a that can, in some embodiments, include a cutting portion 326a on the distal end 334a of the elongate body 322a. The set of arms 354a can be formed, for example, by cutting slits along a wall of the elongate body 322a. The set of arms 354a define an interior volume 356a; the elongate body 322a can, in some embodiments define a lumen 352a, as shown in FIG. 24. In some embodiments, the elongate body does not define a lumen.

The elongate body 322a has an open or expanded configuration, as shown in FIGS. 23A, 23B and 24, and a collapsed or closed configuration, as shown in FIG. 25. The arms 354a can be formed in the same manner and with the same materials as described above for arms 354. The cannula can be used to move the elongate body 322a between the collapsed configuration and the expanded configuration as described above. When the elongate body 322a is in the closed or collapsed configuration, the interior volume 356a defined by the set of arms 354a is smaller than the interior volume 356a defined by the set of arms 354a when the elongate body 322a is in the open or expanded configuration.

FIG. 26 illustrates another medical device according to an embodiment of the invention that can be used in a similar manner as the embodiments described with reference to FIGS. 7-13. In this embodiment, a medical device 420 includes an elongate body 422 having a distal portion 430 and a proximal portion (not shown), and defining a lumen 452. The elongate body 422 also includes a distal end 434 having a cutting portion 426 that defines a pair of openings 448 that have a non-circular shape. Although two openings 448 are shown in FIG. 26, in other embodiments, more than two openings 448 can be included.

In use, the medical device 420 is inserted into a tumor within a vertebra such that a portion of the tumor is disposed within the lumen 452 of the elongate body 422. The elongate body 422 is then rotated such that the cutting portion 426 severs the portion of the tumor from the remaining portion of the tumor within the vertebra. Once the tumor is severed, the cutting portion 426 can be misaligned with respect to the center cut through the tumor by cutting portion 426. This allows the cutting portion 426 to at least partially retain the excised portion of the tumor while being withdrawn from the patient.

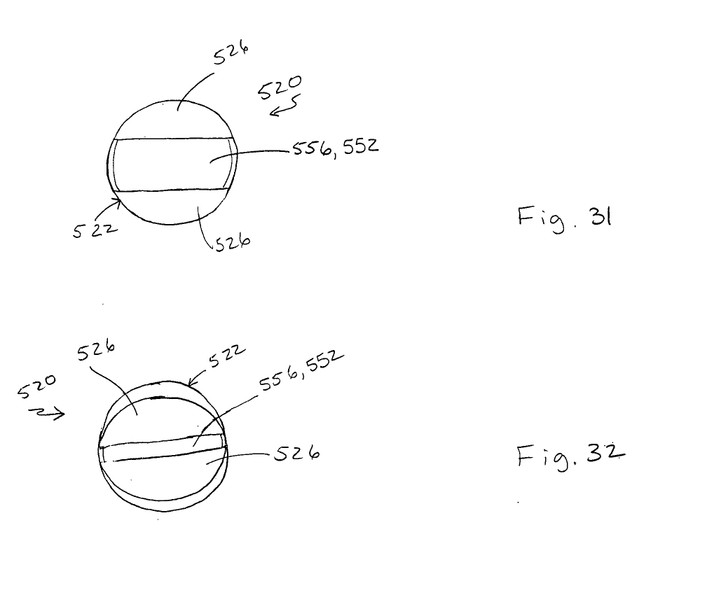

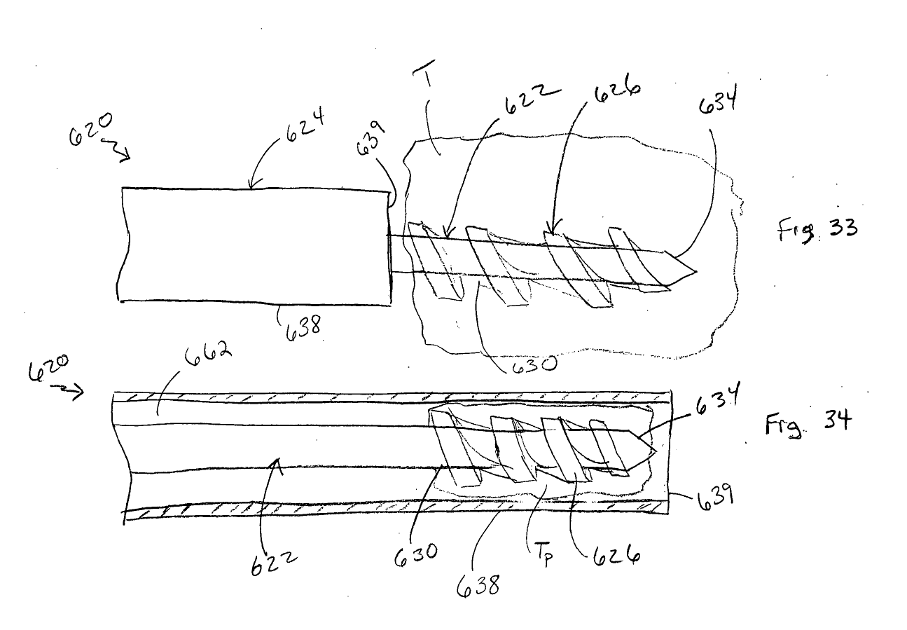

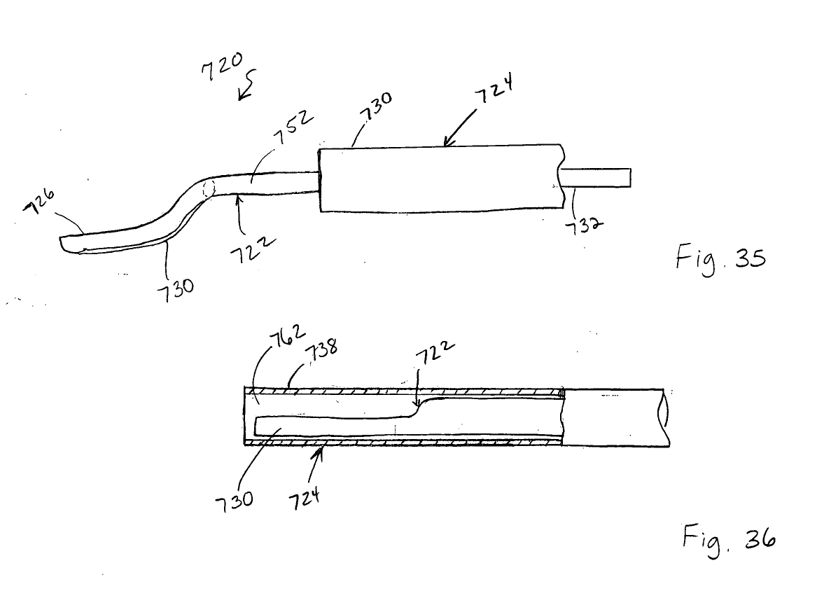

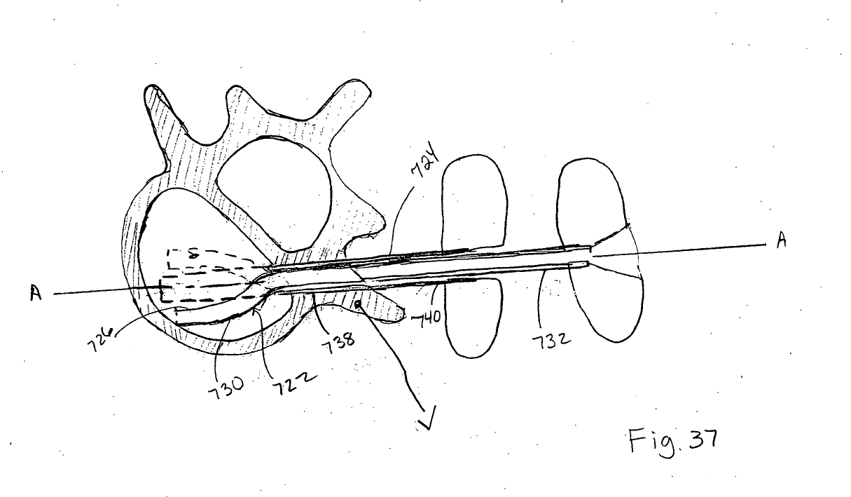

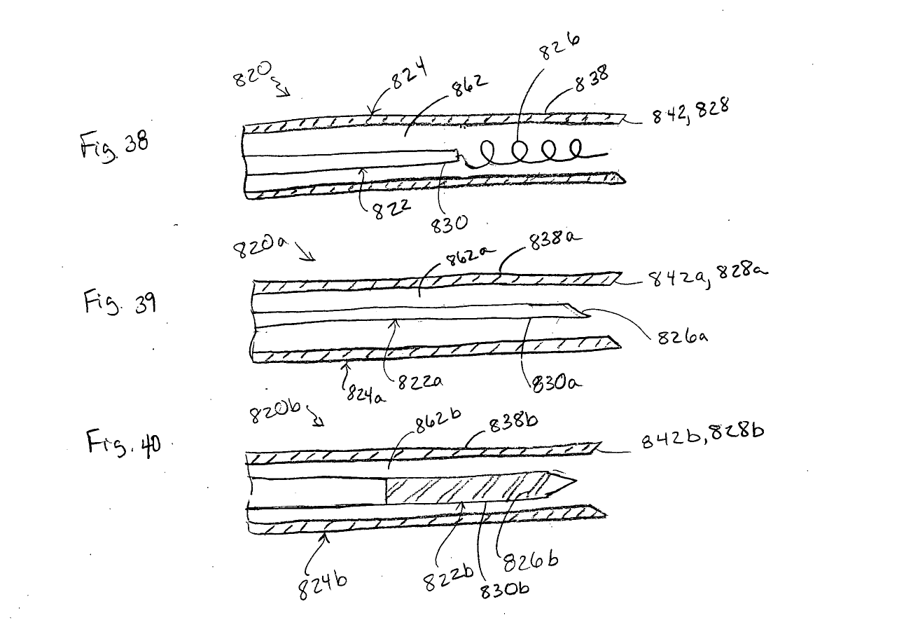

Another similar embodiment of a medical device is illustrated in FIGS. 27 and 28. In this embodiment, a medical device 420a includes an elongate body 422a having a distal portion 430a and a proximal portion (not shown), and defining a lumen 452a. The elongate body 422a also includes a distal end 434a having a cross-member 460a, instead of a cutting portion that defines a pair of openings 448a. In some embodiments, a cutting portion can be included on the cross-member 460a.

In this embodiment, a one-way valve 458a is disposed within the lumen 452a of the elongate body 422a. The one-way valve 458a includes a cutting portion 426a along a peripheral edge of the one-way valve 458a. The one-way valve 458a is constructed of elastic or super-elastic material such that it is biased into a substantially closed configuration, as shown in FIG. 28, and can be moved to an open configuration, as shown in FIG. 27.

In use, the distal portion 430a of the elongate body 422a is inserted into a tumor within a vertebra. When inserted, the one-way valve 458a is pushed open by a portion of the tumor. When the elongate body 422a is pulled out of the tumor (moved proximally), the one-way valve 458a will be biased back to the collapsed configuration and as the one-way valve 458a assumes the collapsed configuration, the cutting portion 426a will sever the portion of the tumor proximate the one-way valve 458a from the remaining portion of the tumor distal to the one-way valve 458a.

The cross member 460a of medical device 420a and the cutting portion 426 of medical device 420 can each be disposed at a distal end of an elongate body or at a location proximal of the distal end of an elongate body and disposed within a lumen of the elongate body. A cutting portion 426 as shown in FIG. 26 and/or a cross member 460a and/or a one-way valve 458a can also be included with any of the embodiments illustrated in FIGS. 7-13. For example, the cross member 460a or cutting portion 426 can be disposed across the opening 248f in FIG. 13.

FIGS. 29 and 30 illustrate a medical device according to another embodiment of the invention in which the medical device can be moved from a closed configuration to an open configuration by heat activation. A medical device 520 includes an elongate body 522 having a proximal portion (not shown) and a distal portion 530 having a distal end 534. The elongate body 522 also defines a lumen 552 between the proximal portion (not shown) and the distal portion 534. The distal portion 530 defines an interior volume 556 and a pair of cutting portions 526 are disposed at the distal end 534 of the elongate body 522.

The elongate body 522 has an open configuration, as shown in FIGS. 29 and 31, and a substantially closed configuration, as shown in FIGS. 30 and 32. When the elongate body 522 is in the substantially closed configuration, the interior volume 556 is smaller than when the elongate body 522 is in the open configuration. The distal portion 530 of the elongate body 522 is constructed with a material that enables the elongate body 522 to be moved between the open configuration and the substantially closed configuration through heat activation. Examples of such materials include shape-memory alloys such as Nitinol. Although only two cutting portions are illustrated in FIGS. 29-32, it is to be understood that in alternative embodiments, more than two cutting portions (e.g., 3, 4, 5 or 6, etc.) can be included.

The elongate body 522 is configured to be inserted into a tumor within a vertebra while in the open configuration such that the cutting portions 526 are cut into the tumor and a portion of the tumor is disposed within the interior volume 556. The body temperature or heat within the vertebra will activate the distal portion 530 of the elongate body 522 and cause the distal portion 530 to move to a substantially closed configuration. When the elongate body 522 is moved from the open configuration to the substantially closed configuration and moved proximally, the cutting portions 526 sever the portion of the tumor from the remaining portion of the tumor and the severed portion of the tumor will be disposed within the interior volume 556. The elongate body 522 can then be removed from the vertebra.

FIGS. 33 and 34 illustrate a medical device according to another embodiment of the invention. In this embodiments, a medical device includes a drill configuration. A medical device 620 includes a cannula 624 and an elongate body 622 movably disposed within a lumen 662 of the cannula 624. The cannula has a proximal portion (not shown) and a distal portion 638 having a distal end 639. The elongate body 622 has a proximal portion (not shown) and a distal portion 630 having a cutting portion 626 in a drill configuration and a sharpened distal end 634. In this embodiment, the medical device 620 has an extended configuration as shown in FIG. 33, in which the distal portion 630 of the elongate body 622 is positioned at a location outside of the cannula 624, and a collapsed configuration as shown in FIG. 34, in which the distal portion 630 of the elongate body 622 is disposed within the lumen 662 of the cannula 624.

The medical device 620 is configured to be inserted into a tumor T within a vertebra (not shown) in the extended configuration and rotated such that the cutting portion 626 cuts or disrupts a portion or portions of the tumor from the remaining portion of the tumor within the vertebra. At least a portion of the cut or disrupted portion or portions of the tumor Tp will be captured by the elongate body 622 and the cutting portion 626. The cannula 624 is then moved or collapsed over the distal portion 630 of the elongate body 622 containing the elongate body 622 with the cut or disrupted portion(s) of the tumor Tp within the lumen 662 of the cannula 624. In some embodiments, the cannula can include a cutting portion (not shown) at a distal end of the cannula. The cutting portion of the cannula can further help sever the cut or disrupted portion(s) of the tumor Tp from the remaining portion of the tumor T when the cannula is moved distally over the distal portion of the elongate body. In some embodiments, the cutting portion 626 anchors the elongate body 622 within the tumor and the cannula is then moved distally over the elongate body such that the cannula severs a portion of the tumor and captures the portion of the tumor within a lumen of the cannula as described above.

FIGS. 35-37 illustrate a medical device according to another embodiment of the invention. In this embodiment, the medical device includes an elongate body that is formed, for example, with a shape-memory or super elastic material to allow it to move from a collapsed configuration when restrained, to an off-center configuration when unrestrained. A medical device 720 includes a cannula or introducer 724 and an elongate body 722 movably disposable within a lumen 762 of the cannula 724. The cannula 724 includes a proximal portion 740 (see FIG. 37) and a distal portion 738. The elongate body 722 includes a proximal portion 732 and a distal portion 730, and defines a lumen (not shown). In some alternative embodiments, the elongate body does not define a lumen. The distal portion 730 of the elongate body 722 has a generally curved shaped forming a scoop-like configuration and includes a cutting portion 726 along at least one peripheral edge of the distal portion 730.

The elongate body 722 is constructed with a deformable material having shape-memory or super elastic characteristics that allow the medical device 720 to be moved between a collapsed configuration, as shown in FIG. 36, in which the distal portion 730 of the elongate body 722 is disposed within the lumen 762 of the cannula 724, and an expanded configuration, as shown in FIGS. 35 and 37, in which the distal portion 730 of the elongate body 722 is located outside of the cannula 724 in an off-set position with respect to a longitudinal axis A defined by the cannula 724.

The medical device 720 can be percutaneously inserted into a vertebra while in the collapsed configuration. The medical device 720 can be moved to the expanded configuration by moving the distal portion 730 of the elongate body 722 distally to a location outside of the cannula 724, as shown in FIG. 35. When the distal portion 730 of the elongate body 722 is moved to this position, the distal portion 730 assumes the off-set position with respect to the longitudinal axis A defined by the cannula 724, as shown in FIG. 35 and FIG. 37 due to the shape-memory characteristics of the elongate body 722. In the expanded configuration, the medical device 720 can be rotated within the vertebra to carve out a portion of a tumor (not shown) within the vertebra, as shown in FIG. 37 by the dotted-line projections of the movement of the distal end 730 within the vertebra. The scoop-like configuration of the distal portion 730 together with the cutting portion 726 can sever or cut the tumor, and hold a portion within the distal portion 730. The medical device 720 can then be moved to the collapsed configuration with the elongate body 722 located within the lumen 762 of the cannula 724 and the severed portion of the tumor within the scoop-like distal portion 730. The medical device 720 can then be removed from the vertebra in the collapsed configuration with the severed portion of the tumor.

FIGS. 38-40 illustrate various embodiments of medical devices according to the invention. In these embodiments, a medical device includes a cutting portion disposed within an interior of an outer body. A medical device 820 (820a, 820b) includes an outer body 824 (824a, 824b) and an elongate body 822 (822a, 822b) disposed within a lumen 862 (862a, 862b) of the outer body 824 (824a, 824b). The outer body 824 (824a, 824b) includes a proximal portion (not shown) and a distal portion 838 (838a, 838b) having a distal end 842 (842a, 842b). The distal end 842 (842a, 842b) includes a cutting portion 828 (828a, 828b) configured to penetrate a tumor within a vertebra.

The elongate body 822 (822a, 822b) includes a proximal portion (not shown) and a distal portion 830 (830a, 830b). The distal portion 830 (830a, 830b) includes a cutting portion 826 (826a, 826b). As shown in FIG. 38. the elongate body 822 has a cutting portion 826 with a corkscrew-type configuration. As shown in FIG. 39, the elongate body 822a has a cutting portion 826a in the form of a sharp distal tip. As shown in FIG. 40, the elongate body 822b has a cutting portion 826b with a drill-type configuration.

In use, the medical device 820 (820a, 802b) can be percutaneously inserted into a tumor within a vertebra such that a portion of the tumor becomes lodged or captured on the distal portion 830 (830a, 830b) of the elongate body 822 (822a, 822b). For example, the medical devices 820 and 820b can be rotated while being inserted within the tumor such that the cutting portions 826 and 826b cut into the tumor and a portion of the tumor becomes attached to, or captured on, the cutting portion 826, 826b and within the lumen 862, 862b of the cannula 824, 824b. The medical device 820, 820b can then be pulled out of the tumor severing the portion of the tumor from the remaining portion of the tumor within the vertebra. Similarly, the cutting portion 826a of the medical device 820a can penetrate the tumor and a portion of the tumor can be severed from the remaining portion of the tumor upon removal of the medical device 820a from the tumor.

FIGS. 41-45 illustrate various embodiments of a medical device according to the invention. In these embodiments, the medical device includes at least one opening on a sidewall of an elongate body that is configured to be inserted into a tissue. A medical device 920, shown in FIGS. 41 and 42, includes an elongate body 922 having a proximal portion (not shown) and a distal portion 930 having a distal end 934. The elongate body 922 also defines a lumen 952. The distal portion 930 defines an opening 948 on a sidewall of the elongate body 920 and an opening 950 disposed at the distal end 934. A cutting portion 926 is disposed along a peripheral edge of the opening 948 and both the opening 948 and the opening 950 are in communication with the lumen 952. In some embodiments, a cutting portion can also be included on a peripheral edge of the opening 950.

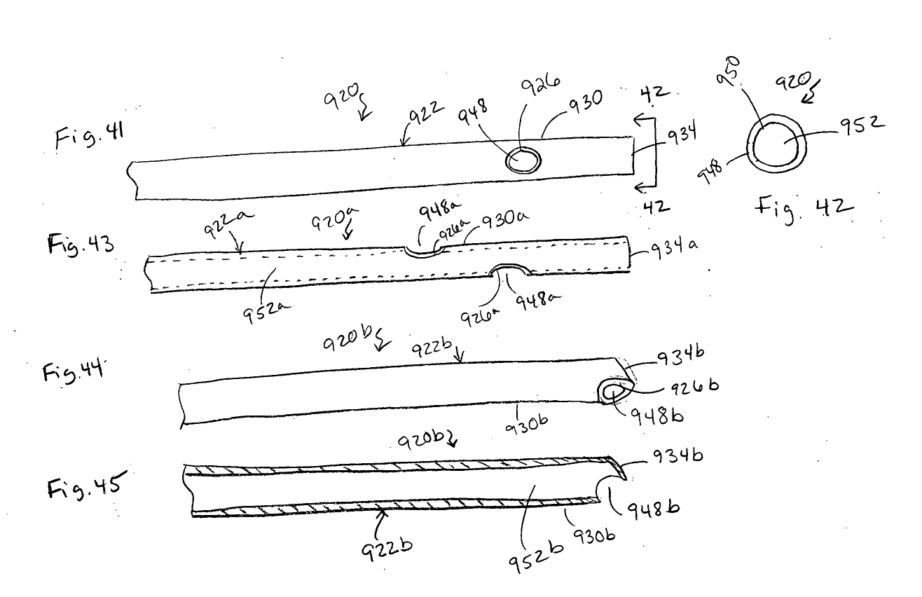

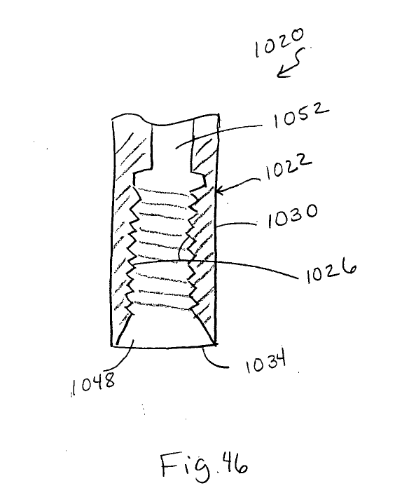

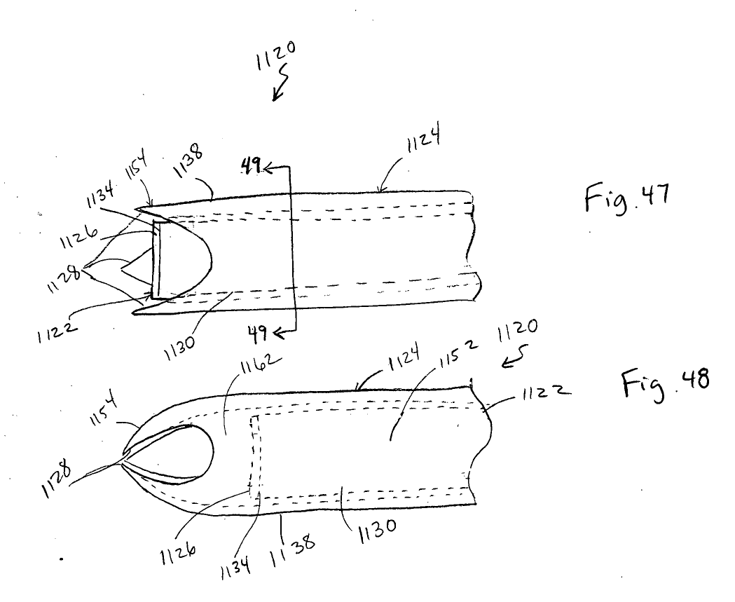

A medical device 920a, shown in FIG. 43, is similar to the medical device 920 except in this embodiment two openings 948a are in communication with a lumen 952a of the elongate body 922a defined on a distal portion 930a of the elongate body 922a. In addition, in this embodiment, the elongate body 922a has a closed distal end 934a. As with medical device 920, a cutting portion 926a is disposed at a peripheral edge of the openings 948a.

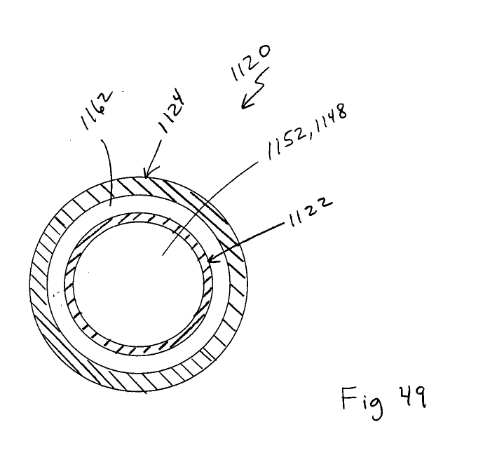

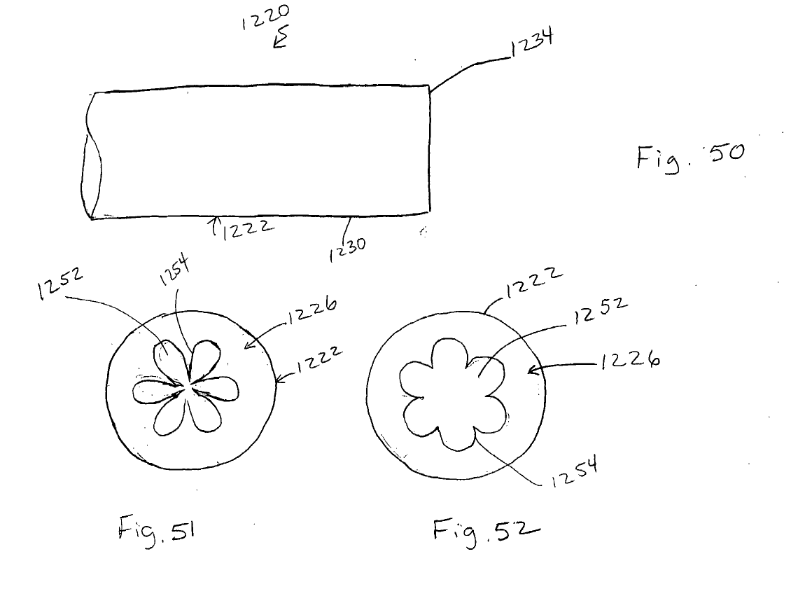

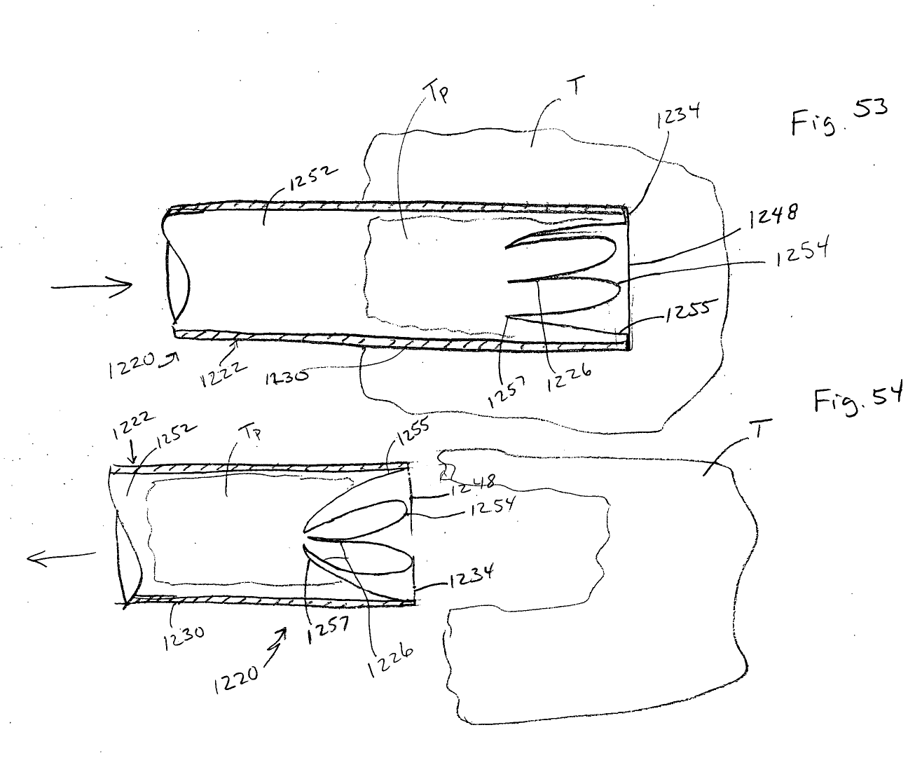

The medical device 920b, shown in FIGS. 44 and 45, is similar to the medical devices 920 and 920a, except in this embodiment, an elongate body 922b includes an opening 948b on a distal end 934b of a distal portion 930b. The opening 948b is in communication with a lumen 952b and a cutting portion 926b is disposed along a peripheral edge of the opening 948b.

In use, each of the medical devices 920, 920a and 920b can be percutaneously inserted into a tumor within a vertebra. The medical device 920 (920a, 920b) can be rotated while inserted within the tumor such that the cutting portion 926 (926a, 926b) severs a portion of the tumor from the remaining portion of the tumor and the severed portion of the tumor is disposed through the opening 948 (948a, 948b) and within the lumen 952 (952a, 952b).

FIG. 46 illustrates a medical device according to another embodiment of the invention. In this embodiment, the medical device includes a cutting portion disposed within an interior of an elongate body in a threaded configuration. A medical device 1020 includes an elongate body 1022 having a proximal portion (not shown), a distal portion 1030, and defining a lumen 1052. An opening 1048 is defined on a distal end 1034 of the elongate body 1022 that is in communication with the lumen 1052. A cutting portion 1026 is disposed at an inner surface of the distal portion 1030 of the elongate body 1022. The cutting portion 1026 has a threaded configuration similar to a tapped hole.

The medical device 1020 can be percutaneously inserted into a tumor within a vertebra and rotated while inserted within the tumor such that the cutting portion 1026 severs a portion of the tumor from the remaining portion of the tumor. The medical device 1020 can then be withdrawn from the tumor with the tumor captured within the lumen 1052 of the elongate body 1022 by the cutting portion 1026.

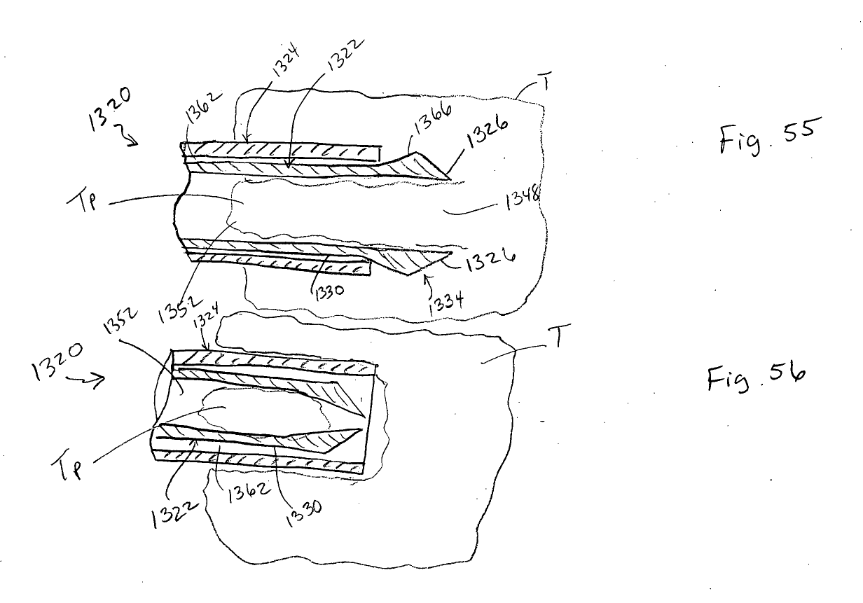

A medical device according to another embodiment of the invention is illustrated in FIGS. 47-49. In this embodiment an outer body of the medical device is formed at least partially with a shape-memory material. A medical device 1120 includes an outer body 1124 and an inner body 1122 movably disposed within a lumen 1162 of the outer body 1124. The inner body 1122 has a proximal portion (not shown) and a distal portion 1130 having a distal end 1134. An opening 1148 is defined on the distal end 1134 that is in communication with a lumen 1152 of the inner body 1122, as shown in FIG. 49. A cutting portion 1126 is disposed at a peripheral edge of the opening 1148.

The outer body 1124 includes a proximal portion (not shown) and a distal portion 1138. The distal portion 1138 of the outer body 1124 includes a plurality of cutting portions 1128 disposed on deformable arms 1154. The deformable arms 1154 are constructed with a flexible or deformable material having shape-memory characteristics such that the deformable arms 1154 are biased into a substantially closed position, as shown in FIG. 48, and can be pushed open by the inner body 1122, as shown in FIGS. 47 and 49. The medical device 1120 has a collapsed configuration when the deformable arms 1154 are in the substantially closed position, and an expanded configuration when the deformable arms 1154 are in the open configuration.

The medical device 1120 can be percutaneously inserted into a vertebra in the collapsed configuration. The medical device 1120 can then be moved from the collapsed configuration to the expanded configuration by moving the distal end 1134 of the inner body 1122 to a location outside of the outer body 1124 and into a tumor within the vertebra. The cutting portion 1126 will cut into the tumor and a portion of the tumor will be disposed within the lumen 1152 of the inner body 1122. The inner body 1122 can then be moved proximally such that the distal portion 1130 of the inner body 1122 is disposed within the lumen 1162 of the outer body 1124. When the distal portion 1130 of the inner body 1122 is moved into the location within the lumen 1162 of the outer body 1124, the deformable arms 1154 on the outer body 1124 will automatically collapse to the substantially closed position, and while doing so, the cutting portions 1128 will sever the portion of the tumor proximate from cutting portions 1128 from the remaining portion of the tumor distal from cutting portions 1128 within the vertebra.

FIGS. 50-54 illustrate another embodiment of a medical device according to the invention. In this embodiment, the medical device includes a cutting portion formed at least partially with a shape-memory material disposed within an interior of an elongate body. A medical device 1220 includes an elongate body 1222 having a proximal portion (not shown) and a distal portion 1230, and defines an opening 1248 on a distal end 1234 that is in communication with a lumen 1252. A cutting portion 1226 is disposed at the distal portion 1230 of the elongate body 1222. The cutting portion 1226 includes arms 1254 constructed with a shape-memory material. A distal end 1255 of the arms 1254 is coupled to the distal end 1134 of the elongate body 1222, and a proximal end 1257 of the arms 1254 extends inwardly from the distal end 1134 of the elongate body into the lumen 1252 of the elongate body 1222. In other embodiments, the cutting portion 1226 may extend inwardly from a location different from the distal end 1134 on the elongate body 1222. For example, the distal end 1255 of the arms 1254 can be coupled to the elongate body 1222 at a location different from the distal end 1134 of the elongate body 1222.

The medical device 1222 has a closed or collapsed configuration in which the arms 1254 are biased into a substantially closed position, as shown in FIGS. 51 and 54, and an open or expanded configuration, in which the arms are moved to an open position, as shown in FIGS. 52 and 53. The medical device 1220 can be percutaneously inserted into a vertebra in the collapsed configuration and into a tumor T within the vertebra, as shown in FIG. 53. As the medical device 1220 is inserted into the tumor T, a portion of the tumor Tp will push open the arms 1254, as shown in FIG. 53. The medical device 1220 can then be pulled out of the tumor T and the arms 1254 will move to the closed position due to the bias of the arms 1254, as shown in FIG. 54. While doing so, the cutting portions 1226 on the arms 1254 will sever the portion of the tumor Tp from the remaining portion of the tumor T within the vertebra, and the portion of the tumor Tp will be captured within the lumen 1252 of the elongate body 1222, as shown in FIG. 54.

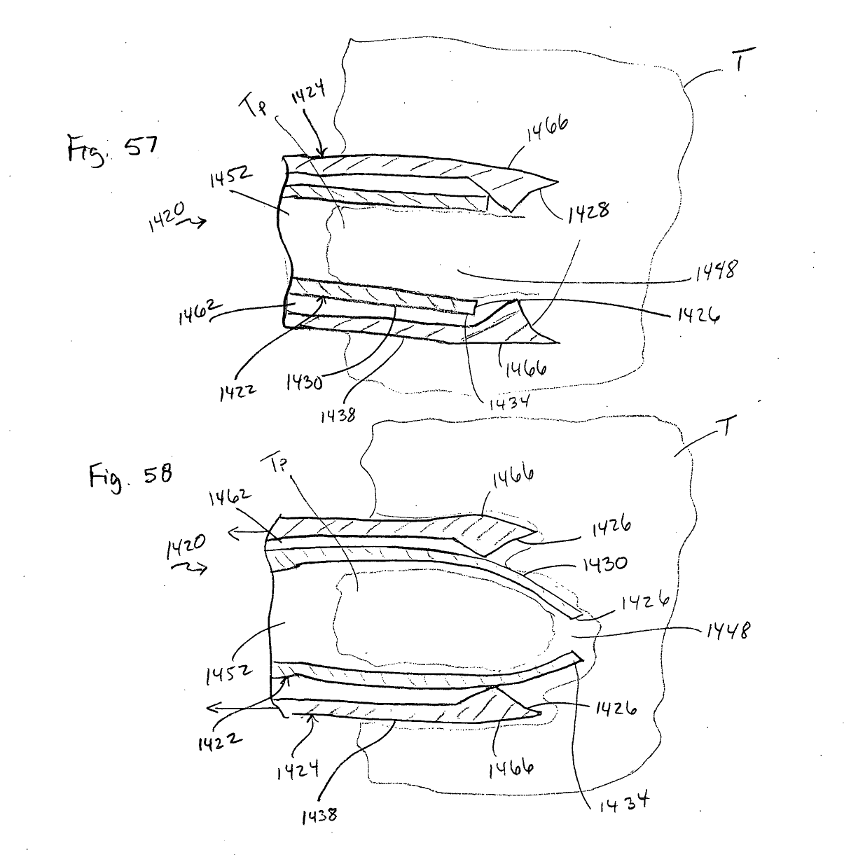

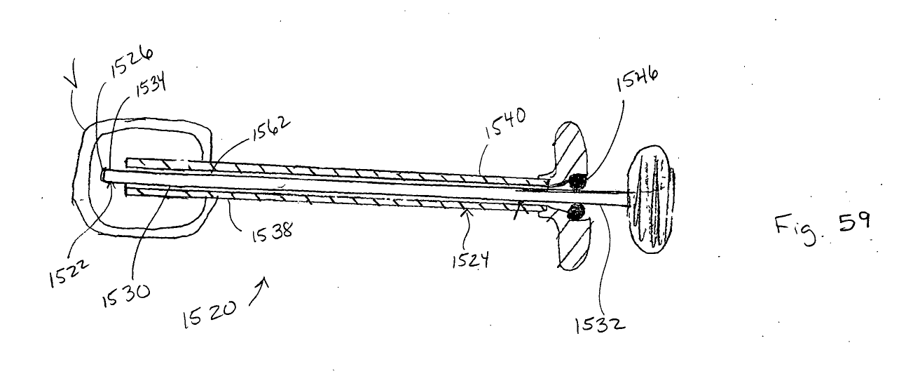

FIGS. 55 and 56 illustrate another medical device according to an embodiment of the invention. In this embodiment, the medical device includes an inner body that is formed with a flexible or deformable material and configured to be moved between a collapsed configuration and an expanded configuration. A medical device 1320 includes an outer body 1324 and an inner body 1322 movably disposed within a lumen 1362 of the outer body 1324. The inner body 1322 includes a proximal portion (not shown) and a distal portion 1330 having a distal end 1334, and defines a lumen 1352. The distal end 1334 of the inner body 1322 defines an opening 1348 in communication with the lumen 1352 of the inner body 1322, and a cutting portion 1326 is disposed at the distal end 1334.

The inner body 1322 is constructed with a flexible or deformable material that allows the distal portion 1330 of the inner body 1322 to be moved from an open position, as shown in FIG. 55, to a substantially closed position, as shown in FIG. 56. The medical device 1320 has an expanded configuration in which the distal portion 1330 of the inner body 1322 is in the open position, and a collapsed configuration in which the distal portion 1330 of the inner body 1322 is in the substantially closed position.

The distal portion 1330 of the inner body 1322 also includes a portion 1366 having a greater wall thickness than the remainder of the inner body 1322 when the medical device 1320 in the expanded configuration. The inner body 1322 also has a greater outer diameter associated with the portion 1366 than the remainder of the distal portion 1366 of the inner body 1322 when the medical device 1320 is in the expanded configuration. In this embodiment, an outer diameter of the inner body 1322 at the portion 1366 is greater than a diameter of the lumen 1362 of the outer body 1324 when the medical device 1320 is in the expanded configuration.

To move the medical device 1320 from the expanded configuration to the collapsed configuration, the inner body 1322 can be moved proximally such that the distal portion 1330 of the inner body 1322 is disposed within the lumen 1362 of the outer body 1324, or the outer body 1324 can be moved distally over the inner body 1322 such that the distal portion 1330 of the inner body 1322 is disposed within the lumen 1362 of the outer body 1324. In either case, as the outer body 1324 contacts the portion 1366 of the inner body 1322, the outer body 1324 will push or force the distal portion 1330 of inner body 1322 to the substantially closed position.

The medical device 1320 can be percutaneously inserted into a tumor T within a vertebra in the expanded configuration such that the cutting portion 1326 penetrates into the tumor T and a portion of the tumor Tp is disposed within the lumen 1352 of the inner body 1322, as shown in FIG. 46. The medical device 1320 can then be moved to the collapsed configuration as described above, such that as the distal portion 1330 is closed, the cutting portion 1326 severs the portion of the tumor Tp from the remaining portion of the tumor T within the vertebra, and the severed portion of the tumor Tp is disposed within the lumen 1352 of the inner body 1322, as shown in FIG. 56.

FIGS. 57 and 58 illustrate another medical device according to an embodiment of the invention. In this embodiment, the medical device includes an inner body that is formed with a flexible or deformable material, and an outer body that can also be formed with a flexible or deformable material. A medical device 1420 includes an outer body 1424 and an inner body 1422 movably disposed within a lumen 1462 of the outer body 1424. The inner body 1422 includes a proximal portion (not shown) and a distal portion 1430, and defines a lumen 1452. The distal portion 1430 defines an opening 1448 in communication with the lumen 1452 of the inner body 1422 and includes a cutting portion 1426 at a distal end 1434.

The outer body 1424 includes a proximal portion (not shown) and a distal portion 1438. The outer body 1424 also includes a portion 1466 on the distal portion 1438 that has a greater wall thickness than the remainder of the distal portion 1438 of the outer body 1424. The outer body 1424 also has a smaller inner diameter associated with the portion 1466 than the remainder of the outer body 1424. The outer body 1424 can also be constructed with a flexible or deformable material.

The inner body 1422 and the outer body 1424 are each constructed with a flexible or deformable material that allows the medical device 1420 to be moved between an expanded or open configuration, as shown in FIG. 57, and a collapsed or substantially closed configuration, as shown in FIG. 58. When the medical device is in the expanded configuration, an inner diameter of the outer body 1422 at the portion 1466 is less than an outer diameter of the inner body 1422.

To move the medical device 1420 from the expanded configuration to the collapsed configuration, the outer body 1424 can be moved proximally such that the portion 1466 of the distal portion 1438 pushes or forces the distal portion 1430 of the inner body 1422 to the substantially closed position.

The medical device 1420 can be percutaneously inserted into a tumor T within a vertebra in the expanded or open configuration such that the cutting portion 1428 of the outer body 1424 penetrates into the tumor T and a portion of the tumor Tp is disposed within the lumen 1452 of the inner body 1422, as shown in FIG. 57. The medical device 1420 can then be moved to the collapsed or substantially closed configuration as described above, such that as the distal portion 1430 is closed, the cutting portion 1426 on the inner body 1422 severs the portion of the tumor Tp from the remaining portion of the tumor T within the vertebra and the portion of the tumor Tp is disposed within the lumen 1452 of the inner body 1422, as shown in FIG. 58.

FIG. 59 illustrates another embodiment of a medical device according to the invention. This embodiment illustrates the use of a seal between an outer body and an inner body of the medical device. In this embodiment, a medical device 1520 includes an outer body 1524 and an inner body 1522 that is movably disposed within a lumen 1562 of the outer body 1524. The inner body 1522 includes a proximal portion 1532, a distal portion 1530, and a cutting portion 1526 disposed at a distal end 1534. The cutting portion 1526 is configured to penetrate a tumor (not shown) within a vertebra V.

The outer body 1524 includes a proximal portion 1540 and a distal portion 1538. This embodiment also includes a seal member 1546 disposed between the outer body 1524 and the inner body 1522 that is configured to block or seal a space between an outer surface of the inner body 1522 and an inner surface of the outer body 1524. Although the seal member 1546 is shown disposed at the proximal portion 1532 of the inner body 1522 and the proximal portion 1540 of the outer body 1524, the seal member 1546 can be disposed at any location along the longitudinal length of the medical device 1520. The seal 1546 illustrated in this embodiment can be included in any of the previously described embodiments having an inner body and an outer body to seal a space between the two components. The seal 1546 can allow a syringe connected to the lumen of the inner body 1522 define a negative pressure within the lumen while disposed inside a vertebra.

The medical device for any of the embodiments may be constructed with any suitable material used for such a medical device. For example, the elongate body or inner body and the cannula or outer body can be constructed with a biocompatible material, such as stainless steel or suitable plastic materials such as various polymers. The cutting member can likewise be constructed with suitable biocompatible metals or plastics. The seal member can be constructed with suitable biocompatible plastic and/or rubber materials.

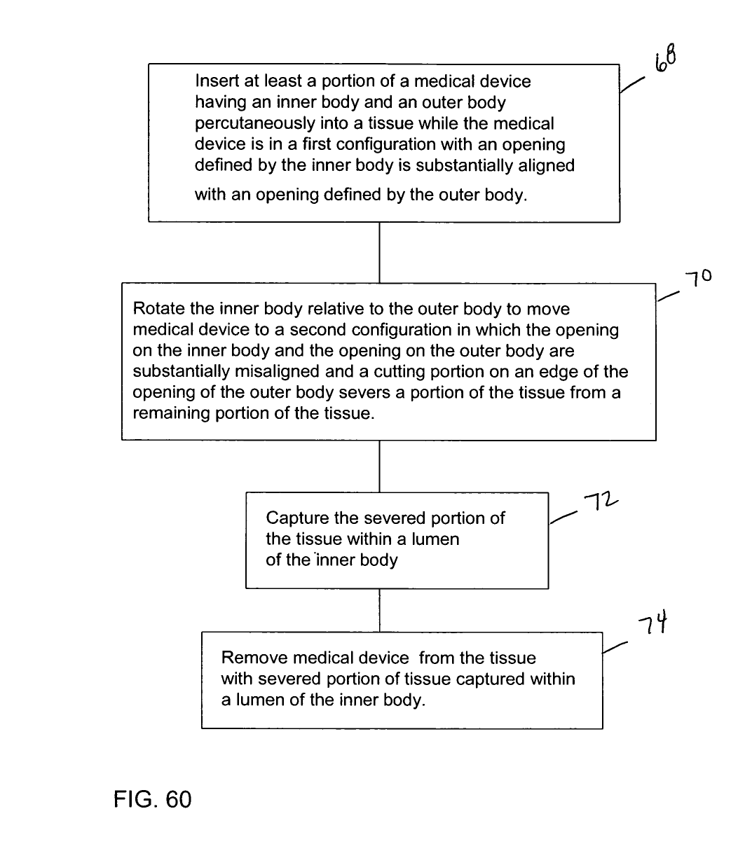

FIG. 60 illustrates a method of performing a medical procedure within a tissue. A method includes at 68, inserting at least a portion of a medical device having an inner body and an outer body percutaneously into a tissue while the medical device is in a first configuration in which an opening defined by the inner body is substantially aligned with an opening defined by the outer body. In some embodiments, the medical device is into a bone structure and in some embodiments, the medical device is inserted into a vertebral body. At 70, the inner body is rotated relative to the outer body such that the medical device is moved to a second configuration in which the opening on the inner body and the opening on the outer body are substantially misaligned and a cutting portion on an edge of the opening of the outer body severs a portion of the tissue from a remaining portion of the tissue. The method can also include at 72, capturing the severed portion of the tissue within a lumen defined by the inner body. At 74, the medical device can be removed from the tissue with the severed portion of the tissue captured within a lumen of the inner body.

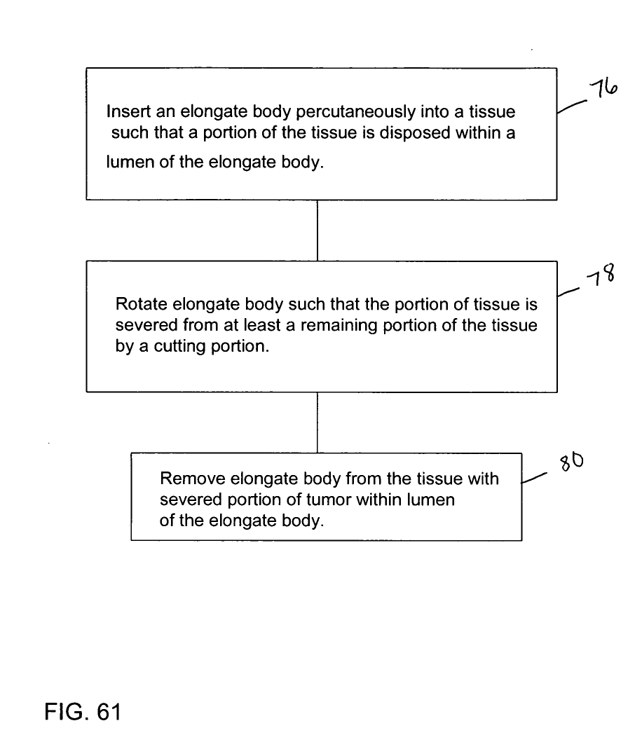

FIG. 61 is a flowchart illustrating a method of performing a medical procedure within a tissue according to another embodiment of the invention. A method includes at 76, inserting an elongate body percutaneously into a tissue such that a portion of the tissue is disposed within a lumen of the elongate body. The elongate body has a cutting portion extending across the lumen and defines a first opening and a second opening. At 78, the elongate body is rotated while inserted into the tissue such that the portion of the tissue is severed from at least a remaining portion of the tissue by the cutting portion. In some embodiments, the elongate body is removed from the tissue with the severed portion of the tumor within the lumen of the elongate body, 80. In some embodiments, the lumen of the elongate body has a non-circular cross-section, and in some embodiments, the first opening and the second opening each have a non-circular cross-section. In some embodiments, the cutting portion extends across the lumen of the elongate body at a distal end of the elongate body.

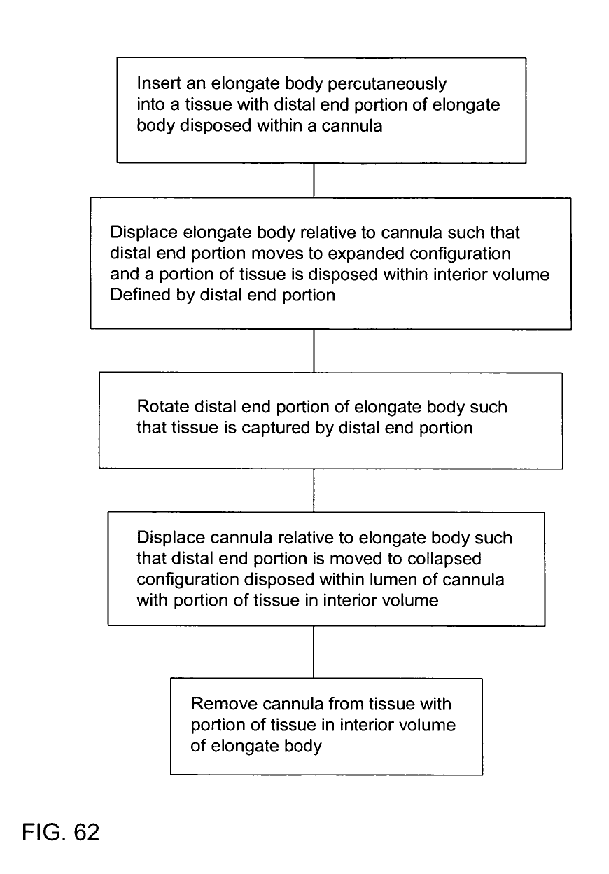

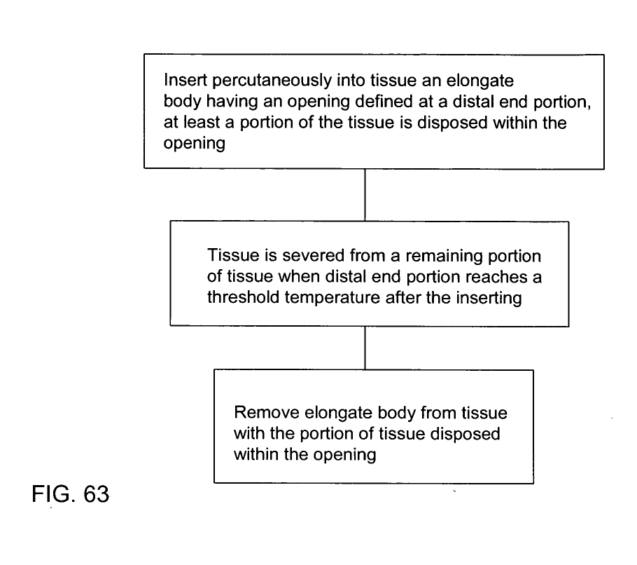

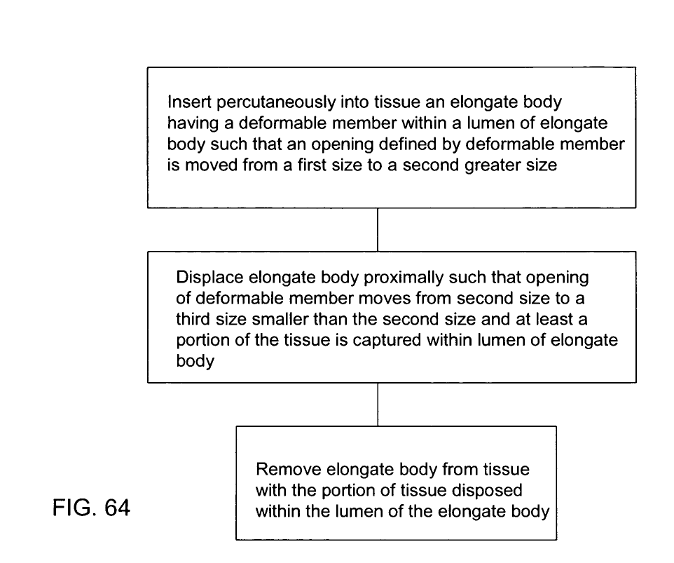

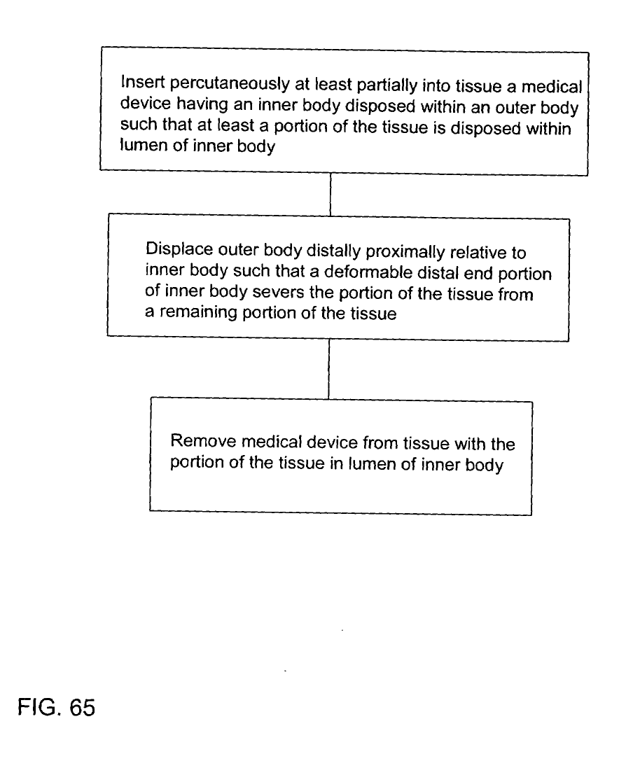

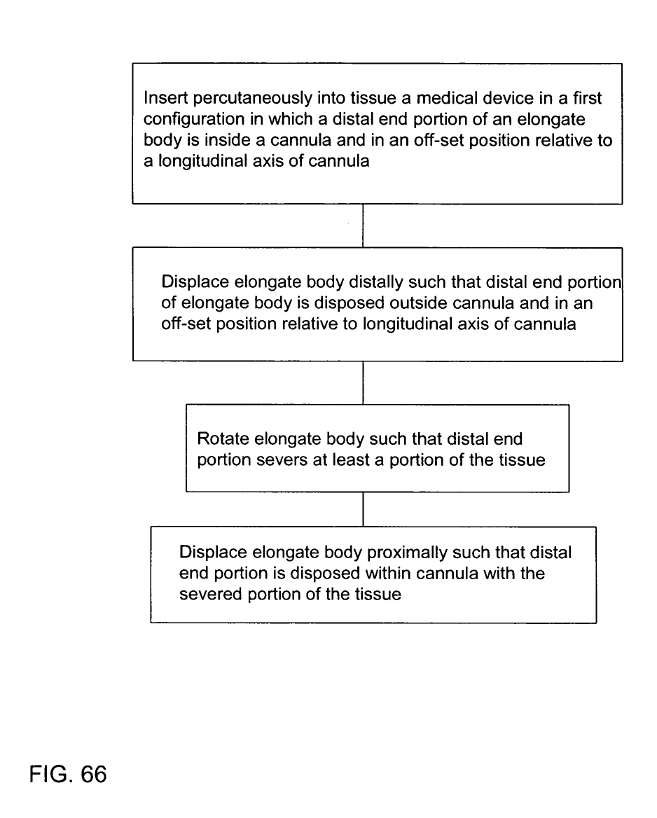

FIGS. 62-66 are each flow charts that illustrate methods of performing a medical procedure within a tissue according to various other embodiments of the invention.

While various embodiments of the invention have been described above, it should be understood that they have been presented by way of example only, and not limitation. It will be understood that various changes in form and details may be made. Where method and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above.

Furthermore, the medical device can include various combinations of the components described in the various embodiments. As stated previously, the medical device is not limited to use within a vertebra and can be used to biopsy a tumor within another bone structure or soft tissue area within a patient's body. In addition, any of the embodiments described above having a single elongate body can be movably disposed within an outer body or cannula. For example, a cannula can be used to provide percutaneous access to a bone structure or soft tissue area of a patient, and the elongate body can be inserted through the cannula and into the bone structure.

What is claimed is:

1. An apparatus, comprising:
an outer body defining an opening at a distal end and a cutting portion disposed at an edge of the opening; and
an inner body movably disposed within the outer body, the inner body defining an opening at a distal end of the inner body,
the outer body and the inner body collectively having a first configuration in which the opening on the outer body is substantially aligned with the opening on the inner body and the opening on the outer body is configured to receive a portion of a tissue, and a second configuration in which the opening on the outer body and the opening on the inner body are substantially misaligned and the cutting portion disposed at the edge of the opening on the outer body is configured to sever the portion of the tissue from a remaining portion of the tissue.

2. The apparatus of claim 1, wherein the portion of the tissue is disposed within a lumen of the inner body when severed from the remaining portion of the tissue.

3. The apparatus of claim 1, wherein the opening on the outer body is a first opening, the outer body defines a second opening on the distal end of the outer body,
the opening on the inner body is a first opening, the inner body defines a second opening on the distal end of the inner body,
the second opening on the inner body is substantially aligned with the second opening on the outer body when the inner body and the outer body are collectively in the first configuration, the second opening on the inner body and the second opening on the outer body are substantially misaligned when the inner body and the outer body are collectively in the second configuration.

4. The apparatus of claim 1, wherein the opening on the outer body is a first opening, the outer body defines a second opening on the distal end of the outer body and a cutting portion disposed at an edge of the second opening, the opening on the inner body is a first opening, the inner body defines a second opening on the distal end of the inner body.

5. The apparatus of claim 1, further comprising:
a seal disposed between the outer body and the inner body.

6. The apparatus of claim 1, wherein the tissue is a bone structure.

7. The apparatus of claim 1, wherein the tissue is a vertebral body.

8. An apparatus, comprising:
an elongate body defining a lumen; and
a cutting portion extending across at least a portion of the lumen of the elongate body to define a first opening and a second opening each in communication with the lumen of the elongate body,
the elongate body configured to be inserted at least partially into a tissue such that a portion of the tissue is cut by the cutting portion and disposed within the lumen of the elongate body,
the elongate body configured to be rotated and moved proximally such that the portion of the tissue is severed from the remaining portion of the tissue.

9. The apparatus of claim 8, wherein the cutting portion is a first cutting portion, the apparatus further comprising:
a one-way valve disposed within the lumen of the elongate body and distally from the first cutting portion; and
a second cutting portion disposed at an edge of the one-way valve,
the one-way valve configured to be moved from a substantially closed configuration to an open configuration when the elongate body is inserted into the tissue,
the one-way valve configured to move from the open configuration to the substantially closed configuration when the elongate body is moved proximally such that the second cutting portion severs a portion of the tissue proximate the one-way valve from a remaining portion of the tissue distal to the one-way valve.

10. The apparatus of claim 8, wherein the cutting portion is disposed at the distal end of the elongate body.

11. The apparatus of claim 8, wherein the first opening and the second opening each have a non-circular cross-section.

12. The apparatus of claim 8, wherein the tissue is a bone structure.

13. The apparatus of claim 8, wherein the tissue is a vertebral body.

14. An apparatus, comprising:
a cannula defining a lumen; and
an elongate body having a collapsed configuration and an expanded configuration, the elongate body configured to be movably disposed within the lumen of the cannula,
the elongate body having a cutting portion at a distal end portion of the elongate body, the cutting portion defining an interior volume in the elongate body,
at least a portion of the cannula configured to be inserted into an interior portion of a tissue when the elongate body is in the collapsed configuration,
the cutting portion of the elongate body configured to capture at least a portion of the tissue within the interior volume of the elongate body when the elongate body is moved from the collapsed configuration to the expanded configuration while within the interior portion of the tissue,
the cutting portion of the elongate body configured to sever the at least a portion of the tissue within the interior volume of the elongate body from a remaining portion of the tissue when the elongate body is moved from the expanded configuration to the collapsed configuration while within the interior of the tissue.

15. The apparatus of claim 14, wherein the cutting portion includes a plurality of arms, each arm from the plurality of arms having a cutting surface on a distal end, the cutting surface configured to cut the portion of the tissue when the elongate body is moved from the expanded configuration to the collapsed configuration while within the interior of the tissue.

16. The apparatus of claim 14, wherein the cannula is configured to be collapsed over the distal portion of the elongate body to move the elongate body from the expanded configuration to the collapsed configuration.

17. The apparatus of claim 14, wherein the elongate body includes a lumen between the distal end portion and a proximal end portion.

18. The apparatus of claim 14, wherein the interior volume is smaller when the elongate body is in the collapsed configuration than when the elongate body is in the expanded configuration.

19. The apparatus of claim 14, further comprising:
a seal disposed between the elongate body and the cannula.

20. A method, comprising:
inserting at least a portion of a medical device having an inner body and an outer body percutaneously into a tissue while the medical device is in a first configuration in which an opening defined by the inner body is substantially aligned with an opening defined by the outer body; and
rotating the inner body relative to the outer body such that the medical device is moved to a second configuration in which the opening on the inner body and the opening on the outer body are substantially misaligned and a cutting portion on an edge of the opening of the outer body severs a portion of the tissue from a remaining portion of the tissue.

21. The method of claim 20, wherein the severed portion of the tissue is captured within a lumen defined by the inner body.

22. The method of claim 20, wherein the inserting the medical device into a tissue includes inserting the medical device into a bone structure.

23. The method of claim 20, wherein the inserting the medical device into a tissue includes inserting the medical device into a vertebral body.

24. The method of claim 20, further comprising:
removing the medical device from the tissue with the severed portion of the tissue captured within a lumen of the inner body.

25. An apparatus, comprising:
an inner body movably disposable within a lumen of an outer body,
the inner body and the outer body collectively configured be inserted into a tissue with an opening defined by the inner body substantially aligned with an opening defined by the outer body,
the inner body and the outer body configured to be rotated relative to each other while inserted into the tissue such that at least a portion of the tissue is severed from a remaining portion of the tissue and disposed within a lumen of the inner body.

26. A method, comprising:
inserting an elongate body percutaneously into a tissue such that a portion of the tissue is disposed within a lumen of the elongate body, the elongate body having a cutting portion extending across the lumen and defining a first opening and a second opening;
rotating the elongate body while inserted into the tissue such that the portion of the tissue is severed from at least a remaining portion of the tissue by the cutting portion.

27. The method of claim 26, further comprising:
removing the elongate body from the tissue with the severed portion of the tumor within the lumen of the elongate body.

28. The method of claim 26, wherein the lumen of the elongate body has a non-circular cross-section

29. The method of claim 26, wherein the first opening and the second opening each have a non-circular cross-section.

30. The method of claim 26, wherein the cutting portion extends across the lumen of the elongate body at a distal end of the elongate body.

31. An apparatus, comprising:
an elongate body having a cutting portion extending across a lumen of the elongate body and configured to be percutaneously inserted into a tissue and rotated such that the cutting portion severs at least a portion of the tissue from the tissue and captures the severed portion of tissue within a lumen of the elongate body.

32. A method comprising:
inserting an elongate body percutaneously into a tissue while a distal end portion of the elongate body is disposed within a cannula;
displacing the elongate body relative to the cannula such that the distal end portion of the elongate body moves to an expanded configuration and a portion of the tissue is disposed within an interior volume defined by the distal end portion of the elongate body; and
after the displacing the, displacing the cannula relative to the elongate body such that the distal end portion of the elongate body is moved to a collapsed configuration disposed within the lumen of the cannula with the portion of the tissue within the interior volume of the distal end portion of the elongate body.

33. The method of claim 32, wherein the displacing the elongate body includes moving the elongate body distally such that the distal end portion of the elongate body is disposed outside the lumen of the cannula.

34. The method of claim 32, further comprising:
removing the cannula from the tissue with the portion of the tissue disposed within the interior volume of the elongate body.

35. The method of claim 32, further comprising:
rotating the distal end portion of the elongate body while inserted into the tissue such that the tissue is captured by the distal end portion of the elongate body.

36. The method of claim 32, wherein the distal end portion of the elongate body includes a cutting portion.

37. The method of claim 32, wherein the tissue is a vertebral body.

38. The method of claim 32, wherein the tissue is a bone structure.

39. An apparatus, comprising:
an elongate body movably disposable within a lumen of a cannula and configured to be percutaenously inserted into a tissue,
the elongate body configured to be moved from a collapsed configuration to an expanded configuration while inserted into the tissue such that a portion of the tissue is disposed within an interior volume defined by the elongate body,
the elongate body configured to be moved to the collapsed configuration while inserted within the tissue such that the portion of the tissue is severed from the tissue and captured within the interior volume of the elongate body.
